SEQUENCE LISTING (50 sequences)

SEQ ID NO: 1 - protein, 126 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKPLYYYDEQYGVVYDAFVWGRGTMVTVSS

SEQ ID NO: 2 - protein, 5 aa, Artificial Sequence
  Description of Artificial Sequence Synthetic peptide
SYAMS

SEQ ID NO: 3 - protein, 17 aa, Artificial Sequence
  Description of Artificial Sequence Synthetic peptide
AISGSGGSTYYADSVKG

SEQ ID NO: 4 - protein, 17 aa, Artificial Sequence
  Description of Artificial Sequence Synthetic peptide
PLYYYDEQYGVVYDAFV

SEQ ID NO: 5 - protein, 111 aa, Artificial Sequence
  Description of Artificial Sequence Synthetic polypeptide
QSVLTQPPSVSGAPGQRVTISCTGSSSNIGAGYDVHWYQQLPGTAPKLLIYGDTHRPSGVPDRFSGSKSGTSASLVIAGLQAEDEADYYCQSYDTVRLHHVFGGGTKLTVL

SEQ ID NO: 6 - protein, 14 aa, Artificial Sequence
  Description of Artificial Sequence Synthetic peptide
TGSSSNIGAGYDVH

SEQ ID NO: 7 - protein, 7 aa, Artificial Sequence
  Description of Artificial Sequence Synthetic peptide
GDTHRPS

SEQ ID NO: 8 - protein, 11 aa, Artificial Sequence
  Description of Artificial Sequence Synthetic peptide
QSYDTVRLHHV

SEQ ID NO: 9 - protein, 126 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKPLYYYDEQYGVVYDAFVWGRGTLVTVSS

SEQ ID NO: 10 - protein, 111 aa, Artificial Sequence
  Description of Artificial Sequence Synthetic polypeptide
QSVLTQPPSVSGAPGQRVTISCTGSSSNIGAGYDVHWYQQLPGTAPKLLIYGDTHRPSGVPDRFSGSKSGTSASLAITGLQAEDEADYYCQSYDTVRLHHVFGGGTKLTVL

SEQ ID NO: 11 - protein, 17 aa, Artificial Sequence
  Description of Artificial Sequence Synthetic peptide
DGASSTNWGYNYYGMDV

SEQ ID NO: 12 - protein, 17 aa, Artificial Sequence
  Description of Artificial Sequence Synthetic peptide
DGASSTNWGYTVDAAVD

SEQ ID NO: 13 - protein, 17 aa, Artificial Sequence
  Description of Artificial Sequence Synthetic peptide
DGASSTNWGYTLDPPGV

SEQ ID NO: 14 - protein, 13 aa, Artificial Sequence
  Description of Artificial Sequence Synthetic peptide
SGSSSNIGSNYVF

SEQ ID NO: 15 - protein, 7 aa, Artificial Sequence
  Description of Artificial Sequence Synthetic peptide
WNNQRPS

SEQ ID NO: 16 - protein, 11 aa, Artificial Sequence
  Description of Artificial Sequence Synthetic peptide
AAWDDSLSGLV

SEQ ID NO: 17 - protein, 11 aa, Artificial Sequence
  Description of Artificial Sequence Synthetic peptide
AAWDDHLEQLH

SEQ ID NO: 18 - protein, 11 aa, Artificial Sequence
  Description of Artificial Sequence Synthetic peptide
AAWDDAARVLL

SEQ ID NO: 19 - protein, 18 aa, Artificial Sequence
  Description of Artificial Sequence Synthetic peptide
PLYYYDGSDYTTYDAFDI

SEQ ID NO: 20 - protein, 18 aa, Artificial Sequence
  Description of Artificial Sequence Synthetic peptide
PLYYYDAPPPLGYDGFDI

SEQ ID NO: 21 - protein, 18 aa, Artificial Sequence
  Description of Artificial Sequence Synthetic peptide
PLYYYDAAPPLGYDGFDI

SEQ ID NO: 22 - protein, 18 aa, Artificial Sequence
  Description of Artificial Sequence Synthetic peptide
PLYYYDAPSPLGYDGFDI

SEQ ID NO: 23 - protein, 18 aa, Artificial Sequence
  Description of Artificial Sequence Synthetic peptide
PLYYYDEQYGLVYDAFDI

SEQ ID NO: 24 - protein, 18 aa, Artificial Sequence
  Description of Artificial Sequence Synthetic peptide
PLYYYDESLALPVYDADI

SEQ ID NO: 25 - protein, 11 aa, Artificial Sequence
  Description of Artificial Sequence Synthetic peptide
QSYDTSLSGSL

SEQ ID NO: 26 - protein, 11 aa, Artificial Sequence
  Description of Artificial Sequence Synthetic peptide
QSYDTAGGGHH

SEQ ID NO: 27 - protein, 11 aa, Artificial Sequence
  Description of Artificial Sequence Synthetic peptide
QSYDTDAARHQ

SEQ ID NO: 28 - protein, 11 aa, Artificial Sequence
  Description of Artificial Sequence Synthetic peptide
QSYDTHLVAHV

SEQ ID NO: 29 - protein, 11 aa, Artificial Sequence
  Description of Artificial Sequence Synthetic peptide
QSYDTLLLAPQ

SEQ ID NO: 30 - protein, 11 aa, Artificial Sequence
  Description of Artificial Sequence Synthetic peptide
QSYDTRADDAH

SEQ ID NO: 31 - protein, 120 aa, Artificial Sequence
  Description of Artificial Sequence Synthetic polypeptide
QVQLVESGGGVVQPGRSLRLSCAASGFTFSNYGMHWVRQAPGKGLEWVAGIWNDGINKYHAHSVRGRFTISRDNSKNTLYLQMNSPRAEDTAVYYCARARSFDWLLFEFWGQGTLVTVSS

SEQ ID NO: 32 - protein, 5 aa, Artificial Sequence
  Description of Artificial Sequence Synthetic peptide
NYGMH

SEQ ID NO: 33 - protein, 17 aa, Artificial Sequence
  Description of Artificial Sequence Synthetic peptide
GIWNDGINKYHAHSVRG

SEQ ID NO: 34 - protein, 11 aa, Artificial Sequence
  Description of Artificial Sequence Synthetic peptide
ARSFDWLLFEF

SEQ ID NO: 35 - protein, 108 aa, Artificial Sequence [sequence omitted]

SEQ ID NO: 36 - protein, 11 aa, Artificial Sequence
  Description of Artificial Sequence Synthetic peptide
RASQSVSSYLA

SEQ ID NO: 37 - protein, 7 aa, Artificial Sequence
  Description of Artificial Sequence Synthetic peptide
DASNRAT

SEQ ID NO: 38 - protein, 10 aa, Artificial Sequence
  Description of Artificial Sequence Synthetic peptide
QQRSNWPPLT

SEQ ID NO: 39 - protein, 120 aa, Artificial Sequence
  Description of Artificial Sequence Synthetic polypeptide
QVQLVESGGGVVQPGRSLRLSCAVSGFTFSNYGMHWVRQAPGKGLEWVAAIWNDGENKHHAGSVRGRFTISRDNSKNTLYLQMNSLRAEDTAVYYCARGRYFDWLLFEYWGQGTLVTVSS

SEQ ID NO: 40 - protein, 8 aa, Artificial Sequence
  Description of Artificial Sequence Synthetic peptide
TFSNYGMH

SEQ ID NO: 41 - protein, 17 aa, Artificial Sequence
  Description of Artificial Sequence Synthetic peptide
AIWNDGENKHHAGSVRG

SEQ ID NO: 42 - protein, 11 aa, Artificial Sequence
  Description of Artificial Sequence Synthetic peptide
GRYFDWLLFEY

SEQ ID NO: 43 - protein, 118 aa, Artificial Sequence
  Description of Artificial Sequence Synthetic polypeptide
EVQLMQSGAEVKKPGESLKISCKGSGYSFSFHWIAWVRQMPGKGLEWMGIIHPGASDTRYSPSFQGQVTISADNSNSATYLQWSSLKASDTAMYFCARQRELDYFDYWGQGTLVTVSS

SEQ ID NO: 44 - protein, 5 aa, Artificial Sequence
  Description of Artificial Sequence Synthetic peptide
FHWIA

SEQ ID NO: 45 - protein, 17 aa, Artificial Sequence
  Description of Artificial Sequence Synthetic peptide
IIHPGASDTRYSPSFQG

SEQ ID NO: 46 - protein, 9 aa, Artificial Sequence
  Description of Artificial Sequence Synthetic peptide
QRELDYFDY

SEQ ID NO: 47 - protein, 107 aa, Artificial Sequence
  Description of Artificial Sequence Synthetic polypeptide
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPLTFGGGTKVEIK

SEQ ID NO: 48 - protein, 11 aa, Artificial Sequence
  Description of Artificial Sequence Synthetic peptide
RASQSIGSSLH

SEQ ID NO: 49 - protein, 7 aa, Artificial Sequence
  Description of Artificial Sequence Synthetic peptide
YASQSFS

SEQ ID NO: 50 - protein, 9 aa, Artificial Sequence
  Description of Artificial Sequence Synthetic peptide
HQSSSLPLT

(19) United States
(12) Patent Application Publication
(10) Pub. No.: US 2013/0149312 A1
(43) Pub. Date: Jun. 13, 2013

(54) COMPOSITIONS AND METHODS FOR TREATING COPD EXACERBATION

(71) Applicants: Donna Finch, Cambridge (GB); Anthony Coyle, Boston, MA (US); Martin Stampfli, Hamilton (CA)

(72) Inventors: Donna Finch, Cambridge (GB); Anthony Coyle, Boston, MA (US); Martin Stampfli, Hamilton (CA)

(73) Assignees: MCMASTER UNIVERSITY, Hamilton, ON (CA); MEDIMMUNE LIMITED, Cambridge (GB)

(21) Appl. No.: 13/641,406

(22) PCT Filed: Apr. 18, 2011
(86) PCT No.: PCT/US11/32910

§ 371 Date: Mar. 4, 2013

Related U.S. Application Data
(60) Provisional application No. 61/325,241, filed on Apr. 16, 2010.
(60) Provisional application No. 61/416,102, filed on Nov. 22, 2010.

(51) Int. Cl. A61K 39/395; A61K 31/56

(52) U.S. Cl. CPC A61K 39/3955; A61K 31/56
USPC 424/142.1; 424/172.1; 424/143.1

(57) ABSTRACT

This disclosure relates to methods of treating exacerbation of chronic obstructive pulmonary disease (COPD) with antibodies and antagonists to interleukin 1 receptor 1 (IL-1R1) or IL-1α.

### CROSS-REFERENCE TO RELATED APPLICATIONS

This application claims the benefit under 35 U.S.C. §119 (e) of U.S. Provisional Application No. 61/325,241 filed Apr. 16, 2010, and U.S. Provisional Application No. 61/416,102 filed Nov. 22, 2010, each of which disclosures are herein incorporated by reference in their entirety.

### REFERENCE TO A SEQUENCE LISTING

The instant application contains a Sequence Listing which has been submitted in ASCII format via EFS-Web and is hereby incorporated by reference in its entirety. Said ASCII copy, created on Apr. 18, 2011, is named MED562PC.txt and is 21,079 bytes in size.

### FIELD OF THE INVENTION

The present disclosure relates to methods of treating chronic obstructive pulmonary disease (COPD) exacerbation using anti-IL-1R1 and anti-IL-1α, antagonists, such as antibodies.

### BACKGROUND OF THE INVENTION

COPD represents a severe and increasing global health problem. By 2020, COPD will have increased from 6^{th} (as it is currently) to the 3^{rd} most common cause of death worldwide. In the United Kingdom, COPD currently accounts for 30,000 deaths annually, whereas in the United States, it is believed to account for up to 120,000 deaths per year (Lopez & Murray 1998). Clinically, COPD is a heterogeneous disease which encompasses two main pathological presentations, aspects of both of which can often be seen in the same patients: chronic obstructive bronchitis with fibrosis and obstruction of small airways, and emphysema with enlargement of airspaces and destruction of lung parenchyma, loss of lung elasticity and closure of small airways (Barnes 2004).

Exacerbations of COPD are of major importance in terms of their prolonged detrimental effect on patients, the acceleration in disease progression and the high healthcare costs (Wedzicha & Donaldson 2003).

Interleukin (IL)-1 is a multifunctional cytokine, which plays a major role in inflammatory responses during immune-mediated diseases and infections. IL-1 is produced from a variety of cell types following stimulation with bacterial products, viruses, cytokines or immune complexes. IL-1 displays autocrine and paracrine activities on a variety of cell types promoting the production of inflammatory mediators such as prostaglandins, nitric oxide, cytokines, chemokines, metalloproteinases and adhesion molecules.

### SUMMARY OF THE INVENTION

COPD exacerbation is a serious complication for COPD patients. There is a need for treatments for exacerbations of COPD (COPD exacerbation). This distinct subset of patients (those with exacerbation or during a period of exacerbation) has increased morbidity and mortality associated with COPD, including increase risk of significant disease progression. One class of such agents are those that bind specifically to IL-1R1 and inhibit binding of IL-1R1 to IL-1α and, optionally, IL-1β. Another class of agents are agents that bind specifically to IL-1α and inhibit IL-1α binding to IL-1R1. In certain embodiments, agents of the disclosure are antagonists. In certain embodiments, agents of the disclosure are antibodies or antibody fragments.

The present disclosure relates to methods of treating COPD exacerbations. In certain embodiments, the disclosure relates to a method of reducing airway inflammation in a patient in need thereof. In certain embodiments, the disclosure relates to a method of increasing lung function in a patient in need thereof.

In a first aspect, the disclosure provides a method of reducing airway inflammation in a patient in need thereof, wherein said patient is a patient having chronic obstructive pulmonary disease (COPD) exacerbation. The method comprises administering to said patient an effective amount of a composition comprising an antibody that specifically binds to IL-1R1. For example, the antibody specifically binds to IL-1R1 and inhibits binding of IL-1R1 to IL-1α. In certain embodiments, the antibody also inhibits binding of IL-1R1 to IL-1beta.

In another aspect, the disclosure provides a method of treating chronic obstructive pulmonary disease (COPD) exacerbation in a patient in need thereof. The method comprises administering to said patient an effective amount of a composition comprising an antibody that specifically binds to IL-1R1. For example, the antibody specifically binds to IL-1R1 and inhibits binding of IL-1R1 to IL-1α. In certain embodiments, the antibody also inhibits binding of IL-1R1 to IL-1beta.

In another aspect, the disclosure provides a method of treating COPD exacerbation in a patient in need thereof, wherein said patient is a patient having COPD exacerbation due to human rhinovirus-induced airway inflammation. The method comprises administering to said patient an effective amount of a composition comprising an antibody that specifically binds to IL-1R1. For example, the antibody specifically binds to IL-1R1 and inhibits binding of IL-1R1 to IL-1α. In certain embodiments, the antibody also inhibits binding of IL-1R1 to IL-1beta.

In another aspect, the disclosure provides a method of treating COPD exacerbation in a patient in need thereof, wherein said patient is a patient having COPD exacerbation due to viral infection. The method comprises administering to said patient an effective amount of a composition comprising an antibody that specifically binds to IL-1R1. For example, the antibody specifically binds to IL-1R and inhibits binding of IL-1R1 to IL-1α. In certain embodiments, the antibody also inhibits binding of IL-1R1 to IL-1beta.

In another aspect, the disclosure provides a method of treating COPD exacerbation in a patient in need thereof, wherein said patient is a patient having COPD exacerbation due to bacterial infection. The method comprises administering to said patient an effective amount of a composition comprising an antibody that specifically binds to IL-1R1. For example, the antibody specifically binds to IL-1R and inhibits binding of IL-1R1 to IL-1alpha. In certain embodiments, the antibody also inhibits binding of IL-1R1 to IL-1beta.

In another aspect, the disclosure provides a method of reducing IL-1α signaling in a patient in need thereof, wherein said patient is a patient having chronic obstructive pulmonary disease (COPD) exacerbation. The method comprises administering to said patient an effective amount of a composition comprising an antibody that specifically binds to IL-1R1 and inhibits binding of IL-1R1 to IL-1α.

Methods of treatment include administration of a single dose, as well as admnistration of more than one dose on a treatment schedule.

The various features listed below apply to any of the foregoing or following aspects (and embodiments) of the disclosure. In certain embodiments, reducing airway inflammation is part of a method of treating COPD exacerbation. In certain embodiments, reducing airway inflammation includes a reduction in neutrophil influx into a lung. In certain embodiments, treating COPD exacerbation comprises reducing airway inflammation. In certain embodiments, treating COPD exacerbation comprises reducing neutrophil influx into a lung.

In certain embodiment an antibody has a molecular weight of greater than or equal to about 25 kilodaltons. In certain embodiments, an antibody has a molecular weight of about 150 kilodaltons.

In certain embodiments, the antibody inhibits binding of IL-1R1 to IL-1α and IL-1β.

In certain embodiments, the antibody is a human antibody. In certain embodiments, the antibody can specifically bind to human IL-1R1. In certain embodiments, the antibody can specifically bind to IL-1R1 from one or more species of non-human primate. In certain embodiments, the antibody does not specifically bind to murine or rodent IL-1R1.

In certain embodiments, the method is part of a therapeutic regimen for treating COPD. In certain embodiments, the therapeutic regimen for treating COPD comprises administration of steroids.

In certain embodiments, COPD exacerbation is caused by bacterial infection, viral infection, or a combination thereof. In certain embodiments, prior to COPD exacerbation, said patient had COPD classified as GOLD stage III or GOLD stage 1V.

In certain embodiments, the antibody specifically binds to IL-1R1 with a K_{D} of 50 pM or less when measure by Biacore™. In certain embodiments, the antibody specifically binds to IL-1R1 with a K_{D} of 300 pM or less when measure by Biacore™.

In certain embodiments, the antibody competes with IL-1Ra for binding to IL-1R1.

In certain embodiments, administration is systemic administration. In certain embodiments, the method does not include intranasal administration of said composition. In certain embodiments, the method does not include intranasal administration of said composition and does not include other forms of local administration of said composition to lung. In certain other embodiments, antagonist is administered via two different routes of administration. Such administration may be at the same time or at different times. For example, in certain embodiments, antagonist is administered systemically (such as intravenously) and intranasally. In other embodiments, antagonist is administered via a systemic route and via a route for localized delivery to the lung.

In another aspect, the disclosure provides a method of reducing airway inflammation in a patient in need thereof, wherein said patient is a patient having chronic obstructive pulmonary disease (COPD) exacerbation, comprising administering to said patient an effective amount of a composition comprising an antibody that specifically binds to IL-1α and inhibits binding of IL-1α to IL-1R1. Similarly, administration of an IL-1alpha antagonist is contemplated.

In another aspect, the disclosure provides a method of treating chronic obstructive pulmonary disease (COPD) exacerbation in a patient in need thereof, comprising administering to said patient an effective amount of a composition comprising an antibody that specifically binds to IL-1α and inhibits binding of IL-1α to IL-1R1. Similarly, administration of an IL-1alpha antagonist is contemplated.

In another aspect, the disclosure provides a method of treating COPD exacerbation in a patient in need thereof, wherein said patient is a patient having COPD exacerbation due to human rhinovirus-induced airway inflammation, comprising administering to said patient an effective amount of a composition comprising an antibody that specifically binds to IL-1alpha and inhibits binding of IL-1alpha to IL-1R1. Similarly, administration of an IL-1 alpha antagonist is contemplated.

In another aspect, the disclosure provides a method of treating COPD exacerbation in a patient in need thereof, wherein said patient is a patient having COPD exacerbation due to viral infection, comprising administering to said patient an effective amount of a composition comprising an antibody that specifically binds to IL-1alpha and inhibits binding of IL-1alpha to IL-1R1. Similarly, administration of an IL-1alpha antagonist is contemplated.

In another aspect, the disclosure provides a method of treating COPD exacerbation in a patient in need thereof, wherein said patient is a patient having COPD exacerbation due to bacterial infection, comprising administering to said patient an effective amount of a composition comprising an antibody that specifically binds to IL-1alpha and inhibits binding of IL-1alpha to IL-1R1. Similarly, administration of an IL-1alpha antagonist is contemplated.

In another aspect, the disclosure provides a method of reducing IL-1α signaling in a patient in need thereof, wherein said patient is a patient having chronic obstructive pulmonary disease (COPD) exacerbation, comprising administering to said patient an effective amount of a composition comprising an antibody that specifically binds to IL-1α and inhibits binding of IL-1α to IL-1R1. Similarly, administration of an IL-1alpha antagonist is contemplated.

Methods of treatment include administration of a single dose, as well as admnistration of more than one dose on a treatment schedule.

The various features listed below apply to any of the foregoing or following aspects (and embodiments) of the disclosure. In certain embodiments, reducing airway inflammation is part of a method of treating COPD exacerbation. In certain embodiments, reducing airway inflammation includes a reduction in neutrophil influx into the lung. In certain embodiments, treating COPD exacerbation comprises reducing airway inflammation. In certain embodiments, treating COPD exacerbation comprises reducing neutrophil influx into a lung.

In certain embodiments, the antibody has a molecular weight of greater than or equal to about 25 kilodaltons. In certain embodiments, the antibody has a molecular weight of approximately 150 kilodaltons.

In certain embodiments, the antibody is a human antibody. In certain embodiments, the antibody can specifically bind to human IL-1α. In certain embodiments, the antibody can specifically bind to IL-1α from one or more species of non-human primate. In certain embodiments, the antibody does not specifically bind to murine IL-1alpha.

In certain embodiments, the method is part of a therapeutic regimen for treating COPD. In certain embodiments, the therapeutic regimen for treating COPD comprises administration of steroids. In certain embodiments, COPD exacerbation is caused by bacterial infection, viral infection, or a combination thereof. In certain embodiments, prior to COPD exacerbation, said patient had COPD classified as GOLD stage III or GOLD stage IV.

In certain embodiments, administration is systemic administration. In certain embodiments, the method does not include intranasal administration of said composition and does not include other forms of local administration of said composition to lung. In certain embodiments, the method does not include intranasal administration of said composition. In certain other embodiments, antagonist is administered via two different routes of administration. Such administration may be at the same time or at different times. For example, in certain embodiments, antagonist is administered systemically (such as intravenously) and intranasally. In other embodiments, antagonist is administered via a systemic route and via a route for localized delivery to the lung.

In another aspect, the disclosure provides a method of treating COPD exacerbation in a patient in need thereof, wherein said patient is a patient having COPD exacerbation due to human rhinovirus-induced airway inflammation. The method comprises administering to said patient an effective amount of a composition comprising an antagonist of IL-1R1 that specifically binds to and inhibits IL-1R1. In certain embodiments, the antagonist of IL-1R1 specifically binds to and inhibits binding of IL-1R1 to IL-1alpha and/or beta. In certain embodiments, antagonism is assessed using any assay described herein.

In another aspect, the disclosure provides a method of treating COPD exacerbation in a patient in need thereof, wherein said patient is a patient having COPD exacerbation due to viral infection. The method comprises administering to said patient an effective amount of a composition comprising an antagonist of IL-1R1 that specifically binds to and inhibits IL-1R1. In certain embodiments, the antagonist of IL-1R1 specifically binds to and inhibits binding of IL-1R1 to IL-1alpha and/or beta. In certain embodiments, antagonism is assessed using any assay described herein.

In another aspect, the disclosure provides a method of treating COPD exacerbation in a patient in need thereof, wherein said patient is a patient having COPD exacerbation due to bacterial infection. The method comprises administering to said patient an effective amount of a composition comprising an antagonist of IL-1R1 that specifically binds to and inhibits IL-1R1. In certain embodiments, the antagonist of IL-1R1 specifically binds to and inhibits binding of IL-1R1 to IL-1alpha and/or beta. In certain embodiments, antagonism is assessed using any assay described herein.

Methods of treatment include administration of a single dose, as well as administration of more than one dose on a treatment schedule.

The various embodiments listed below apply to any of the foregoing or following aspects (and embodiments) of the disclosure. In certain embodiments, the antagonist specifically binds to and inhibits human IL-1R1.

In certain embodiments, the antagonist of IL-1R1 is selected from a human antibody that specifically binds to IL-1R1 and an IL-1Ra. In certain embodiments, the antagonist of IL-1R1 is a recombinant IL-1Ra. In certain embodiments, the antagonist specifically binds to IL-1R1 and inhibits binding of IL-1R1 to IL-1 alpha.

In certain embodiments, treating COPD exacerbation comprises reducing airway inflammation. In certain embodiments, treating COPD exacerbation comprises reducing neutrophil influx into a lung.

In certain embodiments, the antagonist has a molecular weight of greater than or equal to about 25 kilodaltons.

In certain embodiments, the method is part of a therapeutic regimen for treating COPD. In certain embodiments, the therapeutic regimen for treating COPD comprises administration of steroids.

In certain embodiments, the antagonist competes with IL-1Ra for binding to IL-1R1.

In certain embodiments, administration is systemic administration. In certain embodiments, the method does not include intranasal administration of said composition. In certain embodiments, the method does not include intranasal administration of said composition and does not include other forms of local administration of said composition to lung. In certain other embodiments, antagonist is administered via two different routes of administration. Such administration may be at the same time or at different times. For example, in certain embodiments, antagonist is administered systemically (such as intravenously) and intranasally. In other embodiments, antagonist is administered via a systemic route and via a route for localized delivery to the lung.

In certain embodiments, prior to COPD exacerbation, said patient had COPD classified as GOLD stage III or GOLD stage 1V.

The disclosure contemplates all combinations of any of the foregoing aspects and embodiments, as well as combinations with any of the embodiments set forth in the detailed description and examples.

### BRIEF DESCRIPTION OF THE TABLES AND FIGURES

FIG. 1 shows IL-1beta activity is inhibited by IL-1R1 blockade in vitro and in vivo. FIG. 1A shows antibody 6 inhibition of IL-1beta induced IL-6 release in primary human COPD lung fibroblast cells in vitro. FIG. 1B shows Anakinra inhibits IL-1beta induced neutrophil mediated inflammation in the mouse lung. Data shown is total neutrophil counts, quantified from bronchoalveolar lavage (BAL) 4 hours after intratracheal challenge with IL-1beta +/−antibody treatment.

FIG. 2 is a schematic illustrating the tobacco smoke induced lung inflammation model.

FIG. 3 shows that IL-1 beta blockade inhibits tobacco smoke induced lung inflammation. There are four panels showing total cells, neutrophils, macrophages and lymphocytes quantified from bronchoalveolar lavage (BAL) at study endpoint as indicated in the schematic for the various groups in the study, namely saline control groups with room air or cigarette smoke (CS) challenge; isotype control group (MAB005) with cigarette smoke challenge, IL-1R1 antibody (35F5) with cigarette smoke challenge, or anakinra (ALZET osmotic pump) with cigarette smoke challenge.

FIG. 4 shows that IL-1alpha and IL-1beta are expressed in a model of cigarette exposure that induces a neutrophilic inflammatory response that is dependent on the IL-1R1 and independent of caspase-1. (A) Representative images showing expression of IL-1α and β in room air and smoke-exposed mice. Insets represent macrophages from the interstitial space. Total levels of IL-1α (B) and β (C) protein were measured by ELISA from lung homogenates of room air and smoke-exposed animals (n=5 mice per group). Wild-type and either IL-1R1-deficient (n=5 mice per group) (D-F) or caspase-1-deficient (G-I) mice (n=3-6 mice per group) were room air or cigarette smoke exposed. Total cells (D and G), mononuclear cells (E and H), and neutrophils (F and I) were assessed in the broncho-alveolar lavage (BAL) of room air and smoke-exposed mice. Total levels of IL-1α (J) and β (K) protein were measured by ELISA from lung homogenates of room air and smoke-exposed wild-type and caspase-1-deficient mice (n=4-6 mice per group).

FIG. 5 shows that antibody blockade of IL-1alpha but not IL-1beta inhibits cigarette smoke induced-inflammation. Smoke-exposed and room air control mice were either left untreated (No Rx), or administered an isotype antibody (IgG isotype), or either an anti-IL-1α or anti-IL-1β blocking antibody. (A) Neutrophil numbers were enumerated in the broncho-alveolar lavage (BAL) (n=4-5 mice per group). Expression of cxcl-1 (B) and il-1β (D) or cxcl-2, cxcl-10 or cxcl5 (F) transcripts relative to no treatment room air control animals (n=5 mice per group) were assessed by fluidigm array and total protein levels of CXCL-1 (C) and IL-1β (E) were measured using Meso Scale Discovery technology (MSD) (n=10 mice per group).

FIG. 6 shows that the expression pattern of IL-1R1 in smoke-exposed mice mirrors that of COPD patients and is required on radio-resistant non-hematopoietic cells for smoke-induced inflammation. (A) IL-1R1 expression in representative images from room air and smoke-exposed mice. (B) Representative images showing expression of the IL-1R1 as assessed in a lung biopsy obtained from a GOLD III COPD patient. (C) Various chimeric mice (coded as bone marrow donor genotype into recipient genotype) were generated. (D) Neutrophils were enumerated from the broncho-alveolar lavage (BAL) of bone marrow chimeric mice exposed to room air or cigarette smoke (n=5-7 mice per group). Expression of cxcl-1 (E), gm-csf (F), and mmp-12 (G) were measured by fluidigm array (n=6-8 mice per group).

FIG. 7 shows an IL-1R antagonist inhibits LPS mediated inflammatory cell influx into the lung in a murine inhaled LPS model of acute lung inflammation. There are four panels showing total cells, neutrophils, macrophages and lymphocytes quantified from BAL at study endpoint 48 hours after inhaled challenge. Data is shown for groups of animals that received no treatment (naive), vehicle or anakinra (via ALZET pump), and either saline or LPS inhaled challenge.

FIG. 8 shows IL-1R1 blockade reduces human rhinovirus (HRV) induced inflammation in vitro. FIG. 8A shows the study protocol for HRV14 infection and IL-1R1 antagonist treatment of BEAS-2b/H292 (epithelial cell lines) cells. FIG. 8B shows the effect of IL-1R1 antagonist treatment on HRV14 dependent IL-8 release of BEAS-2b/H292 cells. FIG. 8C shows an alternative study design for HRV14 infection and IL-1R1 antagonist treatment of BEAS-2b cells. FIG. 8D shows a dose range of anakinra that reduces HRV-induced IL-8 release by BEAS-2B cells using this protocol. FIG. 8E shows effectiveness of both anakinra and IL-1R1 antibody for reducing IL-8 responses to HRV14 in primary normal human bronchial epithelial (NHBE) cells compared to no effect seen using an isotype control antibody.

FIG. 9 shows IL-1R1 antibody reduces virus induced inflammation in a mouse model of acute rhinovirus-induced lung inflammation. Groups shown are treated either with phosphate buffered saline (PBS), isotype control antibody (MAB005) or anti-IL-1R1 antibody (35F5) intraperitoneally or intranasally with the dose shown, and either PBS, HRV-1b or UV-irradiated HRV1b (UV-HRV1b) intranasally. Cells measured are total cells quantified from BAL at study endpoint 24 hrs after HRV or saline administration. Antibodies or saline were administered 24 hours prior to HRV.

FIG. 10 shows the impact of IL-1R1 receptor blockade or deficiency on smoke, smoke+virus or smoke and viral mimic induced inflammation. FIG. 10A shows the smoke +IL-1R1 antagonist study design in BEAS-2B cells. FIG. 10B shows a dose dependent effect of anakinra on smoke induced IL-8 release. FIG. 10C shows the smoke+virus+IL-1R antagonist (anakinra) study design in BEAS-2B cells. FIG. 10D shows that anakinra inhibits the increased IL-8 release seen when both smoke and virus are used as inflammatory stimulus. FIG. 10E shows that IL-1R1 deficiency in smoke-exposed precision cut lung slices (PCLS) attenuates lung resident responses to viral stimulus. PCLS generated from room air or cigarette smoke-exposed wild-type and IL-1R1-deficient animals were stimulated ex vivo with a viral mimic, polyl:C. Expression of cxcl-1 (left-most graph is panel 10E), cxcl-2 (center graph in panel 10E), and cxcl-5 (right-most graph in panel 10E) relative to room air control mock stimulated PCLS (data not shown) was assessed by real time quantitative RT-PCR (n=7-14 lung slices from 3 independent experiments).

FIG. 11 shows that IL-1R1 deficiency and IL-1alpha antibody blockade attenuates exaggerated inflammation in a model of H1N1 influenza virus infection of smoke-exposed mice. (A-C) Room air or smoke-exposed wild-type or IL-1R1-deficient mice were instilled with vehicle or infected with H1N1 influenza A virus. Five days post infection, total cell number (A), mononuclear cell (B), and neutrophil (C) numbers were enumerated from the broncho-alveolar lavage (BAL) (n=19-20 mice per group). (D-F) Room air and smoke-exposed wild-type mice treated daily with either isotype or IL-1α blocking antibodies were instilled with vehicle or infected with H1N1 influenza A virus. Five days post-infection, total cell numbers (D), mononuclear cell (E), and neutrophil (F) numbers were enumerated in the BAL (n=4-5 mice per group).

FIG. 12 shows IL-1alpha and IL-1beta levels in COPD patients during exacerbation of COPD. Panel A shows IL-1alpha and IL-1beta levels in a COPD patient by sputum measurements during periods of stable or exacerbation of disease. Blue bar-period of exacerbation; red line IL-1alpha and green line IL-1beta. Panel B shows increased IL-1beta levels are associated with bacterial presence in COPD lung.

FIG. 13 shows that IL-1alpha and IL-1beta are increased in the lung of COPD patients. Representative images showing expression of IL-1α (A) and β (B) as assessed in lung biopsies obtained from GOLD stage I/II COPD patients. (C) Positive cells were enumerated from two biopsy samples obtained from each patient (n=5 non-COPD and n=9 COPD GOLD stage I-II patients). Statistical significance was determined using a Generalised Linear Mixed Effect model with negative binomial (adjusted for dispersion) to take into account multiple sampling of the same patient. Whiskers of box plot represent 1-99 percentile. Lung sections from the same biopsy samples were scored for IL-1α (D) and β (E) staining in the epithelium as follows: 0, no staining; 1, occasional staining; 2, marked focal staining; 3, marked diffuse staining. A stratified Wilcoxon Ranksum test was used to compare the frequencies of the staining categories (0, 1, 2, and 3) and represented graphically (size of block is proportional to frequency). There was no significant difference in IL-1α epithelial staining between non-COPD and COPD samples, however IL-1β staining was significantly different for COPD vs non-COPD samples (p<0.0001). Levels of IL-1α and β were measured in sputum samples obtained from patients at enrollment during stable disease (F), at onset of exacerbation (G), and 7 days (H) and 35 days (I) post-exacerbation. IL-1alpha and beta levels were significantly corellated at all visits.

Table 1a lists the amino acid sequences for the CDRs of each of antibodies 1-3. Table 1a discloses SEQ ID NOS 2-3, 11, 2-3, 12, 2-3, 13-15, 14-15, and 14-18, respectively, in order of appearance.
Table 1b lists the amino acid sequences of the CDRs of each of antibodies 4-10. Table 1b discloses SEQ ID NOS 2-3, 19, 2-3, 20, 2-4, 2-3, 21, 2-3, 22, 2-3, 23, 2-3, 24, 6-7, 6-7, 6-7, 6-7, 6-7, 6-7, 6-7, 25-26, 8, and 27-30, respectively, in order of appearance.

### DETAILED DESCRIPTION

### (i) Introduction

Inflammation is well established as a hallmark of COPD which increases during COPD exacerbations (increases during period of exacerbation). However, the molecular mechanisms driving these inflammatory responses are poorly understood. Herein are disclosed methods for reducing airway inflammation and methods of treating COPD exacerbations. In particular, the methods comprise using an antibody that binds IL-1R, inhibiting IL-1alpha and/or IL-1beta. Reduction of airway inflammation can be measured at the microlevel by measuring the reduction of pro-inflammatory meadiators and by-products (e.g. cytokines or influx of inflammatory cells) or at the macrolevel by increased lung function as catagorized by Global Initiative for Chronic Obstructive Lung Disease (GOLD) five-stage classification of COPD severity.

Hypertrophy of smooth muscle, chronic inflammation of airway tissues, and general thickening of all parts of the airway wall can reduce the airway diameter in patients with COPD. Inflammation and edema of the tissue surrounding the airway can also decrease the diameter of an airway. Inflammatory mediators released by tissue in the airway wall may serve as a stimulus for airway smooth muscle contraction. Therapy that reduces the production and release of inflammatory mediators can reduce smooth muscle contraction, inflammation of the airways, and edema. Examples of inflammatory mediators are cytokines, chemokines, and histamine. The tissues which produce and release inflammatory mediators include airway smooth muscle, epithelium, and mast cells. Treatment with the compositions and methods disclosed herein can reduce the ability of airway cells to produce or release inflammatory mediators. The reduction in released inflammatory mediators will reduce chronic inflammation, as well as acute inflammation seen during periods of COPD exacerbations, thereby increasing the airway inner diameter, and may also reduce hyper-responsiveness of the airway smooth muscle.

The IL-1 family of cytokines consists of eleven individual members, four of which, namely IL-1α, IL-1β, IL-18 & IL-1Ra (IL-1 receptor antagonist), have been characterised more fully and linked to pathological processes in a variety of diseases (1). IL-1 exists in two different forms; IL-1α and IL-1β, the products of separate genes. These proteins are related at the amino acid level, IL-1α and IL-1β share 22% homology, with IL-1α and IL-1Ra sharing 18% homology. IL-1β shares 26% homology with IL-1Ra. The genes for IL-1α, IL-1β & IL-1Ra are located on a similar region in human chromosome 2q14 (2, 3).

Both IL-1α and IL-1β are synthesized as 31-kDa precursor peptides that are cleaved to generate 17 kDa mature IL-1α and IL-1β. IL-1β is produced by a variety of cell types including epithelial cells and macrophages. It is released from cells after cleavage by the cysteine protease caspase-1 (IL-1β converting enzyme (ICE) (4)). IL-1α is cleaved by calpain proteases and can remain on the plasma membrane from where it appears to activate cells, via direct cell to cell contact (5). Pro-IL-1α contains a nuclear localization sequence in its amino terminal, which can lead to activation of a variety of cellular pathways (6).

IL-1Ra is a naturally occurring inhibitor of the IL-1 system. It is produced as four different isoforms derived from alternative mRNA splicing and alternative translation initiation. A 17 kDa secreted isoform of IL-1Ra is expressed as variably glycosylated species of 22-25 kDa (7,8) now termed sIL-1Ra. An 18 kDa intracellular isoform is termed icIL-1Ra1 (9). The isoform icIL-1Ra2 is produced by an alternative transcriptional splice from an exon located between the icIL-1Ra1 and sIL-1Ra first exons (10). A third 16 kDa intracellular isoform called icIL-1Ra3 has also been identified (11). KINERET® (also known as anakinra) is a recombinant, nonglycosylated form of the human interleukin-1 receptor antagonist (IL-1Ra). KINERET® differs from native human IL-1Ra in that it has the addition of a single methionine residue at its amino terminus KINERET® consists of 153 amino acids and has a molecular weight of 17.3 kilodaltons. KINERET® is approved for the treatment of moderate to severe active rheumatoid arthritis. Anakinra (referred to herein as anakinra and/or KINERET®) is an example of an IL-1R1 antagonist that antagonizes IL-1R1 signaling. In certain embodiments, the methods of the disclosure include administering an IL-1R1 antagonist, such as anakinra or a similar form of IL-1Ra.

IL-1α and IL-1β exert their biological effects by binding to a transmembrane receptor, IL-1R1 (RefSeq NM_{—}00877 for human IL-1R1), which belongs to the IL-1 receptor family. There are three members of the IL-1 receptor family; IL-1 Receptor 1 (IL-1R1 (80 kDa), IL-1RII (68 kDa) and IL-1 receptor accessory protein (IL-1RacP). IL-1R1 and IL1RacP form a complex in the cell membrane to generate a high affinity receptor capable of signalling upon binding of IL-1α or IL-1β. IL-1Ra binds IL-1R1 but does not interact with IL-1RAcP. IL-1α, IL-1β and IL-1Ra also bind IL-Rh which does not have an intracellular signalling domain.

IL-1R1 is termed the signalling receptor as upon ligand binding and complexing with IL-1RAcP signal transduction is initiated via its cytoplasmic tail of 213 amino acid residues (12). Current literature suggests that IL-1RII acts only as a ‘decoy receptor’ either at the cell surface or extracellularly as a soluble form (13). Modulating binding of IL-1R1 to IL-1α and/or IL-1β is a methodology for modulating IL-1 signaling.

IL-1 signaling has an important role in many chronic inflammatory diseases. In certain embodiments, the disclosure comprises inhibiting IL-1 signaling (as part of a treatment for COPD exacerbation) by administering an IL-1R1 antibody that specifically binds to IL-1R1 and inhibits IL-1R1 activity by, at least inhibiting binding to, at least, IL-1α. In certain embodiments, the antibody also inhibits binding of IL-1R1 to IL-1β. In certain embodiments, the disclosure comprises inhibiting IL-1 signaling (as part of a treatment for COPD exacerbation) by administering an IL-1R1 antagonist (an antagonist of IL-1R1). The foregoing IL-1R1 antibodies are examples of such antagonists of IL-1R1. Other examples include anakinra and naturally occurring forms of IL-1Ra. In certain embodiments, the disclosure comprises inhibiting IL-1 signaling (as part of a treatment for COPD exacerbation) by administering an IL-1α antibody that specifically binds to IL-1α and inhibits binding of IL-1α to IL-1R1.

Additional features of these methods and the compositions that can be used in these methods are described herein.

(ii) Terminology

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The numbering of amino acids in the variable domain, complementarity determining region (CDRs) and framework regions (FR), of an antibody follow, unless otherwise indicated, the Kabat definition as set forth in Kabat et al. Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insertion (residue 52a according to Kabat) after residue 52 of H2 and inserted residues (e.g. residues 82a, 82b, and 82c, etc according to Kabat) after heavy chain FR residue 82. The Kabat numbering of residues may be determined for a given antibody by alignment at regions of homology of the sequence of the antibody with a “standard” Kabat numbered sequence. Maximal alignment of framework residues frequently requires the insertion of “spacer” residues in the numbering system, to be used for the Fv region. In addition, the identity of certain individual residues at any given Kabat site number may vary from antibody chain to antibody chain due to interspecies or allelic divergence.

As used herein, the terms “antibody” and “antibodies”, also known as immunoglobulins, encompass monoclonal antibodies (including full-length monoclonal antibodies), polyclonal antibodies, multispecific antibodies formed from at least two different epitope binding fragments (e.g., bispecific antibodies), human antibodies, humanized antibodies, camelised antibodies, chimeric antibodies, single-chain Fvs (scFv), Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies of the disclosure), intrabodies, and epitope-binding fragments of any of the above. In particular, antibodies include immunoglobulin molecules and immunologically active fragments of immunoglobulin molecules, i.e., molecules that contain at least one antigen-binding site. Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., Glm(f, z, a or x), G2m(n), G3m(g, b, or c), Am, Em, and Km(1, 2 or 3)). Antibodies may be derived from any mammal, including, but not limited to, humans, monkeys, pigs, horses, rabbits, dogs, cats, mice, etc., or other animals such as birds (e.g. chickens). In certain embodiments, an antibody may be further described based on its molecular weight. In certain embodiments, the molecular weight is greater than or equal to 25 kilodaltons. In certain embodiments, the antibody is a full length antibody comprising a constant region.

As used herein, the term “antagonist” refers to a compound that inhibits a biological activity. For example an IL-1R1 antagonist is an antagonist of IL-1R1 signaling. For example, a compound that binds to IL-1R1 and inhibits IL-1α and/or IL-1β signaling via IL-1R1 is an IL-1R1 antagonist. A neutralizing antibody, such as an antibody that specifically binds to IL-1R1 and inhibits binding of IL-1R1 to IL-1α and/or IL-1β is an example of an IL-1R1 antagonist. IL-1Ra compounds, such as anakinra, are another example of IL-1R1 antagonists. In certain embodiments, the antagonist can be a protein. In certain embodiments, the antagonist can be a non-polypeptide antagonist, such as a nucleic acid or small molecule.

An antibody inhibits binding of a ligand to a receptor when an excess of antibody reduces the quantity of ligand bound to receptor by at least 50%, 60% or 80%, and more usually greater than about 85% (as measured in an in vitro competitive binding assay).

As used herein, the term “airway” means a part of or the whole respiratory system of a subject that is exposed to air. “Airways” therefore include the upper and lower airway passages, which include but are not limited to the trachea, bronchi, bronchioles, terminal and respiratory bronchioles, alveolar ducts and alveolar sacs. Airways include sinuses, nasal passages, nasal mucosum and nasal epithelium. The airway also includes, but is not limited to throat, larynx, tracheobronchial tree and tonsils.

As used herein the term “IL-1R1” means interleukin 1 receptor 1. The nucleic acid and amino acid sequences of human IL-1R1 are publicly available (RefSeq NM_{—}000877). In some embodiments IL-1R1 may be human or cynomolgus monkey IL-1R1. As described elsewhere herein, IL-1R1 may be recombinant, and/or may be either glycosylated or unglycosylated.

As used herein the term “IL-1α” or “IL-1 alpha” means interleukin 1α. The nucleic acid and amino acid sequences of human IL-1α are publicly available (RefSeq NM_{—}000575.3). In some embodiments IL-1α may be human or cynomolgus monkey IL-1α. As described elsewhere herein, IL-1α may be recombinant, and/or may be either glycosylated or unglycosylated.

As used herein the term “IL-1β” or “IL-1beta” means interleukin 1β. The nucleic acid and amino acid sequences of human IL-1β are publicly available (RefSeq NM_{—}000576). In some embodiments IL-1β may be human or cynomolgus monkey IL-1β. As described elsewhere herein, IL-1β may be recombinant, and/or may be either glycosylated or unglycosylated.

As used herein the term “Geomean” (also known as geometric mean), refers to the average of the logarithmic values of a data set, converted back to a base 10 number. This requires there to be at least two measurements, e.g. at least 2, preferably at least 5, more preferably at least 10 replicate. The person skilled in the art will appreciate that the greater the number of replicates the more robust the geomean value will be. The choice of replicate number can be left to the discretion of the person skilled in the art.

As used herein the term “monoclonal antibody” refers to an antibody from a substantially homogeneous population of antibodies that specifically bind to the same epitope. The term “mAb” refers to monoclonal antibody.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

As used herein, the term “exacerbation” refers to a worsening of symptoms of COPD, relative to a patient's baseline condition. In certain embodiments, a COPD exacerbation may be defined as an event in the natural course of the disease characterized by a change in the patient's baseline lung function, dyspnea, cough, and/or sputum that is beyond normal day-to-day variations, is acute in onset and may warrant a change in medication in a patient with underlying COPD. In certain embodiments, exacerbation of COPD may be an abrupt increase in symptoms of shortness of breath and/or wheezing, and/or increase in production of purulent sputum (sputum containing pus).

(iii) Antibodies and Antagonists

The presently disclosed methods of treating COPD exacerbation comprise administering compositions comprising antagonists and/or antibodies that bind to IL-1R1 or IL-1α. In certain embodiments, antagonists may be protein, nucleic acid or small molecules that bind to and inhibit a target, in some cases preventing binding by other ligands.

In certain embodiments, antibodies for use in the claimed methods are IL-1R1 antibodies that bind to and inhibit IL-1R1 (US Publication No. 20040097712; and US20100221257, each herein incorporated by reference). In certain embodiments, the antibody specifically binds to IL-1R1, such as human IL-1R1. In certain embodiments, the antibody binds to IL-1R1 and inhibits binding of IL-1R1 to IL-1α and/or IL-1β. In certain embodiments, the antibody is a human antibody. In certain embodiments, the antibody binds to the same epitope as antibody 6 or competes with antibody 6 for binding to IL-1R1. In certain embodiments, the antibody competes with IL-1Ra for binding to IL-1R1. In certain embodiments, antibodies of the disclosure do not compete with IL-1Ra for binding to IL-1R1.

By way of example, exemplary human antibodies that specifically bind to IL-1R1 are provided herein. The amino acid sequencees of the CDRs for these human antibodies are set forth in Tables 1a and 1b. The amino acid sequence of the VH and VL of one of these antibodies (antibody 6), and a germlined version thereof, are provided herein. An exemplary rodent antibody that specifically binds to IL-1R1 is the commercially available 35F5 antibody from BD Pharmingen/BD Biosciences.

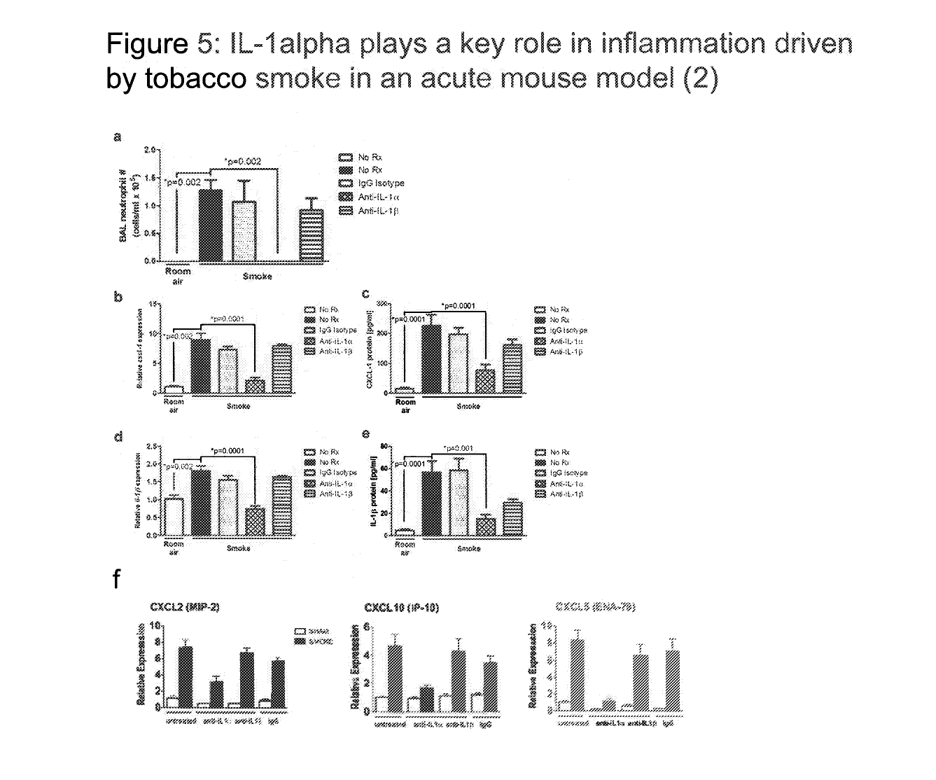

In another embodiment, exemplary human antibodies include those disclosed in US Publication No. 20040097712, including 26F5, 27F2 and 15C4 as disclosed in FIGS. 5, 6, 7, 8, 9, 10 and 11 of US 20040097712, those figures are specifically incorporated by reference. The amino acid sequences for these antibodies are provided herein.

These and other antibodies that specifically bind IL-1R1 and inhibit binding to IL-1alpha and/or IL-1beta are exemplary of IL-1R1 antibodies useful in the present methods. Such antibodies are also examples of IL-1R1 antagonists.

Further exemplary IL-1R1 antagonists include anakinra or other forms of IL-1Ra.

Further antagonists of IL-1R1 or IL-1α that may be suitable for use in the methods of the disclosure have been disclosed in at least the following International Patent Applications: WO2004/022718; WO 2005/023872; WO 2007/063311; WO 2007/063308; WO2005/086695; WO1995/014780 and WO 2006/059108.

In certain embodiments, compounds for use in the claimed methods specifically bind IL-1α and inhibit binding of IL-1α to IL-1R1. An exemplary compound is an antibody that binds specifically to IL-1alpha, such as the commercially available antibody ALF161 from R&D Systems (cat number MAB4001).

Exemplary features that may describe antibodies and antagonists for use in the claimed methods are described below.

In another embodiment, an antibody or antagonist for use in the claimed methods has a mean IC₅₀, of less than 1 nM for the inhibition of IL-1β induced IL-6 production in whole human blood in the presence of 30 pM IL-1β. In further embodiments the mean IC₅₀ is less than 800 pM, less than 700 pM, less than 600 pM, less than 500 pM, less than 400 pM, less than 300 pM, less than 200 pM or less than 100 pM.

Antagonists (antibodies or non-antibody antagonists) of the disclosure bind to IL-1R1 or IL-1α and neutralise IL-1R1 or IL-1α with, for example, high potency. Neutralisation means inhibition of a biological activity of IL-1R1 or IL-1α. Antagonists of the disclosure may neutralise one or more biological activities of IL-1R1, typically antagonists for use in the claimed methods inhibit IL1α and IL1β binding to IL-1R1.

In certain embodiments, the antibody or antagonist specifically binds to and inhibits human IL-1R1. In certain embodiments, the antibody or antagonist specifically binds to and inhibits human IL-1alpha. In certain embodiments, the antibody or antagonist may also bind to and neutralize non-human IL-1R1 or IL-1α, meaning IL-1R1 or IL-1α orthologs that occur naturally in species other than human. In certain embodiments, the non-human species is one or more species of non-human primate, such as cynomolgous.

Binding specificity may be determined or demonstrated, for example, in a standard competition assay.

Suitable assays for measuring neutralisation of IL-1R1 or IL-1α include, for example, ligand receptor biochemical assays and surface plasmon resonance (SPR) (e.g., BIACORE™).

Binding kinetics and affinity (expressed as the equilibrium dissociation constant K_{D}) of IL-1R1 or IL-1α antibodies and antagonists may be determined, e.g. using surface plasmon resonance (BIACORE™). Antibodies and antagonists of the disclosure normally have an affinity (K_{D}) for IL-1R1 or IL-1α, such as human IL-1R1 or IL-1α, of less than about 1 nM, and in some embodiments have a K_{D} of less than about 500 pM, 400 pM, 300 pM, 250 pM, 200 pM, 100 pM, in other embodiments have a K_{D} of less than about 50 pM, in other embodiments have a K_{D} of less than about 25 pM, in other embodiments have a K_{D} of less than about 10 pM, in other embodiments have a K_{D} of less than about 1 pM.

A number of methodologies are available for the measurement of binding affinity of an antibody or antagonist to its antigens, one such methodology is KinExA. The Kinetic Exclusion Assay (KinExA) is a general purpose immunoassay platform (basically a flow spectrofluorimeter) that is capable of measuring equilibrium dissociation constants, and association and dissociation rate constants for antigen/antibody interactions. Since KinExA is performed after equilibrium has been obtained, it is an advantageous technique to use for measuring the K_{D} of high affinity interactions where the off-rate of the interaction may be very slow. The use of KinExA is particularly appropriate in this case where the affinity of antibody and antigen are higher than can be accurately predicted by surface plasmon resonance analysis. The KinExA methodology can be conducted as described in Drake et al (2004) Analytical Biochemistry 328, 35-43.

In one embodiment of the disclosure the antibody or antagonists of the disclosure are specific for IL-1R1 with a K_{D} of 300 pM or lower as measured using the KinExA methodology. Alternatively, a K_{D} of 200 pM or lower, 100 pM or lower, 50 pM or lower, 20 pM or lower, or a K_{D} of 10 pM or lower or 1 pM or lower.

Inhibition of biological activity may be partial or total. Antagonists may inhibit an IL-1R1 biological activity, such as IL-1β induced IL-8 release in CYNOM-Kl cells or IL-1α and IL-1β induced IL-8 release in HeLa cells, by 100%, or alternatively by: at least 95%, at least 90%, at least 85%, at least 80%, at least 75%, at least 70%, at least 60%, or at least 50% of the activity of a concentration of IL-1α or 13 that induces 50% or 80% of the maximum possible activity in absence of the antagonist. Antagonists may inhibit an IL-1α biological activity, such as IL-1α induced IL-8 release in HeLa cells, by 100%, or alternatively by: at least 95%, at least 90%, at least 85%, at least 80%, at least 75%, at least 70%, at least 60%, or at least 50% of the activity of a concentration of IL-1α that induces 50% or 80% of the maximum possible activity in absence of the antagonist.

The neutralising potency of an antagonist is normally expressed as an IC₅₀ value, in nM unless otherwise stated. In functional assays, IC₅₀ is the concentration of an antagonist that reduces a biological response by 50% of its maximum. In ligand-binding studies, IC₅₀ is the concentration that reduces receptor binding by 50% of maximal specific binding level. IC₅₀ may be calculated by plotting % of maximal biological response as a function of the log of the antagonist concentration, and using a software program, such as Prism (GraphPad Software Inc., La Jolla, Calif., USA) to fit a sigmoidal function to the data to generate IC₅₀ values. Potency may be determined or measured using one or more assays known to the skilled person and/or as described or referred to herein. The neutralising potency of an antagonist can be expressed as a geomean.

In certain embodiments, neutralisation of IL-1R1 or IL-1α activity by an antagonist is demonstrated using an assay described herein or any standard assay that indicates that the antagonist binds to and neutralises IL-1R1 or IL-1α. Other methods that may be used for determining binding of an antagonist to IL-1R1 or IL-1α include ELISA, Western blotting, immunoprecipitation, affinity chromatography and biochemical assays.

An antagonist of the disclosure for use in the claimed methods may have a similar or stronger affinity for human IL-1R1 or IL-1α than for IL-1R1 or IL-1α of other species. Affinity of an antagonist for human IL-1R1 or IL-1α may be similar to or, for example, within 5 or 10-fold that for cynomolgus monkey IL-1R1 or IL-1α.

An antagonist of the disclosure for use in the claimed methods comprises, in certain embodiments, an IL-1R1 binding motif comprising one or more CDRs, e.g. a ‘set of CDRs’ within a framework. A set of CDRs comprises one or more CDRs selected from: HCDR1, HCDR2, HCDR3, LCDR1, LCDR2 and LCDR3 (where H refers to heavy chain and L refers to light chain). In one embodiment a set of CDRs comprises a HCDR3 set forth in table 1a or 1 b, optionally combined with one or more CDRs selected from: HCDR1, HCDR2, LCDR1, LCDR2 and LCDR3, as set forth in table 1a or 1b. In another embodiment a set of CDRs comprises a HCDR3 and a LCDR3 set forth in table 1a or 1b, optionally combined with one or more CDRs selected from: HCDR1, HCDR2, LCDR1 and LCDR2, for example one or more CDRs selected from: HCDR1, HCDR2, LCDR1 and LCDR2, as set forth in table 1a or 1b. In another embodiment a set of CDRs comprises a HCDR1, HCDR2, HCDR3, LCDR1, LCDR2 and LCDR3 set forth in table 1a or 1b.

In certain embodiments, an antibody for use in the claimed methods is an antibody having CDRs, as shown in Table 1a. Briefly, a human parent antibody molecule was isolated having the set of CDR sequences as shown in Table 1a (see Antibody 1). Through a process of optimisation, a panel of human antibody clones numbered 2-3, with CDR sequences derived from the parent CDR sequences and having modifications at the positions indicated in Table 1a, was generated. Thus, for example, it can be seen from Table 1a that Antibody 2 has the parent HCDR1, HCDR2, LCDR1 and LCDR2, and has a parent HCDR3 sequence in which: Kabat residue 100E is replaced with T, Kabat residue 100F is replaced with V, Kabat residue 100G is replaced with D, Kabat residue 100H is replaced with A, Kabat residue 100I is replaced with A, Kabat residue 101 is replaced with V and Kabat residue 102 is replaced with D.

In certain embodiments, an antibody for use in the claimed methods is an antibody having CDRs, as shown in Table 1b. Briefly, a second parent human antibody molecule was isolated having the set of CDR sequences as shown in Table 1b (see Antibody 4). Through a process of optimisation, a panel of human antibody clones numbered 5-10 with CDR sequences derived from the parent CDR sequences and having modifications at the positions indicated in Table 1b was generated. Thus, for example, it can be seen from Table 1b that Antibody 5 has the parent HCDR1, HCDR2, LCDR1 and LCDR2, and has a parent HCDR3 sequence in which: Kabat residue 100A is replaced with A, Kabat residue 100B is replaced with P, Kabat residue 100C is replaced with P, Kabat residue 100D is replaced with P, Kabat residue 100E is replaced with L, Kabat residue 100F is replaced with G and Kabat residue 100I is replaced with G.

In certain embodiments, an antibody or antagonist for use in the claimed methods is a human antibody having one or more (1, 2, 3, 4, 5, or 6) CDRs as set forth in Table 1a or 1b. In certain embodiments, an antibody for use in the claimed methods is a human antibody having CDRs as set forth in Table 1a or 1b, wherein one or more of the CDRs have one or more amino acid additions, substitutions, deletions, and/or insertions. For example, in certain embodiments such antibodies have one to five (1, 2, 3, 4, or 5) additions, substitutions, deletions and/or insertions relative to the parent sequences of Antibody 1 or Antibody 4, and retain the ability to specifically bind IL-1R1.

In certain embodiments, the antibody or antagonist has the CDRs of Antibody 6. In certain embodiments, the antibody is a germlined version of Antibody 6. In certain embodiments, the antibody comprises the VH and/or VL of Antibody 6 or a germlined version thereof. Amino acid sequences for antibody 6 and a germlined version of antibody 6 are provided herein. In certain embodiments, the antibody binds the same or substantially the same epitope as antibody 6. In certain embodiments, the antibody competes with antibody 6 for binding to IL-1R1.

In certain embodiments, the antibody or antagonist comprises an Antibody 1 HCDR3 with one or more of the following substitutions or deletions:

Kabat residue 100E replaced by T;

Kabat residue 100F replaced V or L;

Kabat residue 100G replaced by D;

Kabat residue 100H replaced by A or P;

Kabat residue 100I replaced by A or P;

Kabat residue 101 replaced by V or G;

Kabat residue 102 replaced by D or V.

In certain embodiments, the antibody or antagonist comprises an Antibody 4 HCDR3 with one or more of the following substitutions or deletions:

Kabat residue 100A replaced by A or E;

Kabat residue 100B replaced P, Q, or A;

Kabat residue 100C replaced by P, Y, S or L;

Kabat residue 100D replaced by P, G or A;

Kabat residue 100E replaced by L or V;

Kabat residue 100F replaced by G, V or P;

Kabat residue 100G replaced by V;

Kabat residue 100H replaced by Y;

Kabat residue 100I replaced by G or D;

Kabat residue 100J replaced by A or deleted;

Kabat residue 101 replaced by F;

Kabat residue 102 replaced by V.

In certain embodiments, the antibody or antagonist comprises the Antibody 1 LCDR3 with one or more of the following substitutions:

Kabat residue 94 replaced by H or A;

Kabat residue 95 replaced by A;

Kabat residue 95A replaced by E or R;

Kabat residue 95B replaced by Q or V;

Kabat residue 97 replaced by H or L.

In some embodiments, the antibody or antagonist may comprise the Antibody 4 LCDR3 with one or more of the following substitutions:

Kabat residue 94 replaced by A, V, D, H, L or R;

Kabat residue 95 replaced by G, R or A;

Kabat residue 95A replaced by G, L, A, V or D;

Kabat residue 95B replaced by H, R, A or D;

Kabat residue 96 replaced by H, P or A.

Kabat residue 97 replaced by H, V or Q.

In certain embodiments, the antibody or antagonist comprises an Antibody 6 HCDR3 with one or more of the following substitutions or additions:

Kabat residue 100A replaced by G or A;

Kabat residue 100B replaced S, P or A;

Kabat residue 100C replaced by D, P, S or L;

Kabat residue 100D replaced by Y, P or A;

Kabat residue 100E replaced by T or L;

Kabat residue 100F replaced by T, G or P;

Kabat residue 100G replaced by V;

Kabat residue 100H replaced by Y;

Kabat residue 100I replaced by G or D;

Kabat residue 100J deleted in Antibody 6 is reinstated as a A or F;

Kabat residue 101 replaced by D;

Kabat residue 102 replaced by I.

In some embodiments, the antibody or antagonist comprises the Antibody 6 LCDR3 with one or more of the following substitutions:

Kabat residue 94 replaced by S, A, D, H, L or R;

Kabat residue 95 replaced by L, G or A;

Kabat residue 95A replaced by S, G, A, V or D;

Kabat residue 95B replaced by G, R, A or D;

Kabat residue 96 replaced by S, P or A.

Kabat residue 97 replaced by L, H or Q.

In certain embodiments, an antagonist for use in the claimed methods may be one that competes or cross-competes for binding to IL-1R1 with IL-1Ra and/or with an antibody having CDRs set forth in Tables 1a and 1b. In certain embodiments, an antagonist for use in the claimed methods is one that binds the same epitope as an antibody having CDRs set forth in Table 1a and 1b. In certain embodiments, an antagonist for use in the claimed methods is one that binds the same epitope as antibody 6 or an antibody comprising the CDRs of antibody 6. Competition between antagonists may be assayed easily in vitro, for example using ELISA and/or by tagging a specific reporter molecule to one antagonist which can be detected in the presence of one or more other untagged antagonists, to enable identification of antagonists which bind the same epitope or an overlapping epitope. Such methods are readily known to one of ordinary skill in the art, and are described in more detail herein.

In certain embodiments, an IL-1R1 or IL-1alpha antibody for use in the claimed methods is a human, chimeric or humanized antibody. The antibodies may be monoclonal antibodies, especially of human, murine, chimeric or humanized origin, which can be obtained according to the standard methods well known to the person skilled in the art. In certain embodiments, the antagonist is a non-antibody antagonist.

In certain embodiments, an IL-1R1 antagonist for use in the claimed methods is an antibody comprising a VH domain having at least 60, 70, 80, 85, 90, 95, 98 or 99% amino acid sequence identity with a VH domain of antibody 6, or comprising a set of HCDRs (e.g., HCDR1, HCDR2, and/or HCDR3) shown in Table 1a or 1b. The antibody molecule may optionally also comprise a VL domain that has at least 60, 70, 80, 85, 90, 95, 98 or 99% amino acid sequence identity with a VL domain of antibody 6, or with a set of LCDRs (e.g., LCDR1, LCDR2, and/or LCDR3) shown in Table 1a or 1b. Algorithms that can be used to calculate % identity of two amino acid sequences include e.g. BLAST [14], FASTA [15], or the Smith-Waterman algorithm [16], e.g. employing default parameters.

In certain embodiments, an IL-1R1 antagonist for use in the claimed methods is an antibody comprising a VH domain of human antibody 26F5, 27F2, or 15C4 and/or a VL domain of human antibody 26F5, 27F2, or 15C4. In certain embodiments, the antagonist is a human antibody comprising a VH and VL domain of human antibody 26F5 or a VH and VL domain of human antibody 27F2, or a VH and VL domain of human antibody 15C4. In other embodiments, the IL-1R1 antagonist for use in the claimed methods is an antibody comprising 1, 2, 3, 4, 5, or 6 CDRs of human antibody 26F5, 27F2, or 15C4. In certain embodiments, an IL-1R1 antagonist for use in the claimed methods is an antibody comprising a VH domain having at least 80%, 85%, 90%, 95%, 99%, or 99% identity to that of human antibody 26F5, 27F2, or 15C4 and/or a VL domain having at least 80%, 85%, 90%, 95%, 99%, or 99% identity to that of human antibody 26F5, 27F2, or 15C4.

Antibodies for use in the claimed methods may further comprise antibody constant regions or parts thereof, e.g. human antibody constant regions or parts thereof. For example, a VL domain may be attached at its C-terminal end to antibody light chain constant domains including human Cκ or Cλ chains. Similarly, an antagonist based on a VH domain may be attached at its C-terminal end to all or part (e.g. a CH1 domain) of an immunoglobulin heavy chain derived from any antibody isotype, e.g. IgG, IgA, IgE and IgM and any of the isotype sub-classes, particularly IgG1, IgG2, IgG3 and IgG4. IgG1 is advantageous due to its ease of manufacture and stability, e.g., half-life. Any synthetic or other constant region variants which modulate antagonist function and/or properties e.g. stabalizing variable regions, may also be useful in the present disclosure.

Furthermore, it may be desired according to the present disclosure to modify the amino acid sequences described herein, in particular those of human heavy chain constant regions to adapt the sequence to a desired allotype, e.g. an allotype found in the Caucasian population.

In certain embodiments, the antibody may include framework regions of human germline gene sequences, or be non-germlined. Thus, the framework may be germlined where one or more residues within the framework are changed to match the residues at the equivalent position in the most similar human germline framework. Thus, an antagonist for use in the claimed methods may be an isolated human antibody molecule having a VH domain comprising a set of HCDRs in a human germline framework, e.g. human germline IgG VH framework. The antagonist may also have a VL domain comprising a set of LCDRs, e.g. in a human germline IgG VL framework.

In certain embodiments, the antibody may comprise replacing one or more amino acid residue(s) with a non-naturally occurring or non-standard amino acid, modifying one or more amino acid residue into a non-naturally occurring or non-standard form, or inserting one or more non-naturally occurring or non-standard amino acid into the sequence. Examples of numbers and locations of alterations in sequences are described elsewhere herein. Naturally occurring amino acids include the 20 “standard” L-amino acids identified as G, A, V, L, I, M, P, F, W, S, T, N, Q, Y, C, K, R, H, D, E by their standard single-letter codes. Non-standard amino acids include any other residue that may be incorporated into a polypeptide backbone or result from modification of an existing amino acid residue. Non-standard amino acids may be naturally occurring or non-naturally occurring. Several naturally occurring non-standard amino acids are known in the art, such as 4-hydroxyproline, 5-hydroxylysine, 3-methylhistidine, N-acetylserine, etc. [17]. Those amino acid residues that are derivatised at their N-alpha position will only be located at the N-terminus of an amino-acid sequence. Normally, an amino acid is an L-amino acid, but it may be a D-amino acid. Alteration may therefore comprise modifying an L-amino acid into, or replacing it with, a D-amino acid. Methylated, acetylated and/or phosphorylated forms of amino acids are also known, and amino acids in the present disclosure may be subject to such modification.

In certain embodiments, the antibodies used in the claimed methods are generated using random mutagenesis of one or more selected VH and/or VL genes to generate mutations within the entire variable domain. Such a technique is described by Gram et al. [18], who used error-prone PCR. In some embodiments one or two amino acid substitutions are made within an entire variable domain or set of CDRs.

Another method that may be used is to direct mutagenesis to CDR regions of VH or VL genes. Such techniques are disclosed by Barbas et al. [19] and Schier et al. [20].

All the above-described techniques are known as such in the art and the skilled person will be able to use such techniques to provide antagonists of the disclosure using routine methodology in the art.

In certain embodiments, an antibody or antagonist for use in the claimed methods is an antibody fragment. Examples of fragments includie (i) the Fab fragment consisting of VL, VH, constant light chain domain (CL) and constant heavy chain domain 1 (CH1) domains; (ii) the Fd fragment consisting of the VH and CH1 domains; (iii) the Fv fragment consisting of the VL and VH domains of a single antibody; (iv) the dAb fragment [21, 22, 23], which consists of a VH or a VL domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site [24, 25]; (viii) bispecific single chain Fv dimers (for example as disclosed in WO 1993/011161) and (ix) “diabodies”, multivalent or multispecific fragments constructed by gene fusion (for example as disclosed in WO94/13804 and [26]). Fv, scFv or diabody molecules may be stabilized by the incorporation of disulphide bridges linking the VH and VL domains [27]. Minibodies comprising a scFv joined to a CH3 domain may also be made [28]. Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

Suitable fragments may, in certain embodiments, be obtained from any of the human or rodent antibodies disclosed herein. In other embodiments, suitable fragments are obtained from human or rodent antibodies that bind the same epitope of any of the antibodies described herein or that compete for binding to antigen with any such antibodies.

In certain embodiments, antibodies or antagonists for use in the claimed methods are labelled, modified to increase half-life, and the like. For example, in certain embodiments, the antibody or antagonist is chemically modified, such as by PEGylation, or by incorporation in a liposome.

In certain embodiments, an antagonist for use in the claimed methods may comprise an antigen-binding site within a non-antibody molecule, normally provided by one or more CDRs e.g. a set of CDRs in a non-antibody protein scaffold, as discussed further below.

An antigen binding site may be provided by means of arrangement of CDRs on non-antibody protein scaffolds, such as fibronectin or cytochrome B etc. [29, 30, 31], or by randomising or mutating amino acid residues of a loop within a protein scaffold to confer binding specificity for a desired target. Scaffolds for engineering novel binding sites in proteins have been reviewed in detail by Nygren et al. [31]. Protein scaffolds for antibody mimics are disclosed in WO200034784, which is herein incorporated by reference in its entirety, in which the inventors describe proteins (antibody mimics) that include a fibronectin type III domain having at least one randomised loop. A suitable scaffold into which to graft one or more CDRs, e.g. a set of HCDRs, may be provided by any domain member of the immunoglobulin gene superfamily. The scaffold may be a human or non-human protein. An advantage of a non-antibody protein scaffold is that it may provide an antigen-binding site in a scaffold molecule that is smaller and/or easier to manufacture than at least some antibody molecules. Small size of an antagonist may confer useful physiological properties, such as an ability to enter cells, penetrate deep into tissues or reach targets within other structures, or to bind within protein cavities of the target antigen. Use of antigen binding sites in non-antibody protein scaffolds is reviewed in Wess, 2004 [32]. Typical are proteins having a stable backbone and one or more variable loops, in which the amino acid sequence of the loop or loops is specifically or randomly mutated to create an antigen-binding site that binds the target antigen. Such proteins include the IgG-binding domains of protein A from S. aureus, transferrin, tetranectin, fibronectin (e.g. 10th fibronectin type III domain), lipocalins as well as gamma-crystalline and other Affilin™ scaffolds (Scil Proteins). Examples of other approaches include synthetic “Microbodies” based on cyclotides—small proteins having intra-molecular disulphide bonds, Microproteins (Versabodies™, Amunix Inc, Mountain View, Calif., USA) and ankyrin repeat proteins (DARPins, Molecular Partners AG, Zürich-Schlieren, Switzerland). Such proteins also include small, engineered protein domains such as, for example, immuno-domains (see for example, U.S. Patent Publication Nos. 2003/082630 and 2003/157561). Immuno-domains contain at least one complementarity determining region (CDR) of an antibody.

In certain embodiments, antagonists may comprise other amino acids, e.g. forming a peptide or polypeptide, such as a folded domain, or to impart to the molecule another functional characteristic in addition to ability to bind antigen. Antagonists may carry a detectable label, or may be conjugated to a toxin or a targeting moiety or enzyme (e.g. via a peptidyl bond or linker).

In certain embodiments, the half-life of an antagonist or antibody for use in the claimed methods is at least about 4 to 7 days. In certain embodiments, the mean half-life is at least about 2 to 5 days, 3 to 6 days, 4 to 7 days, 5 to 8 days, 6 to 9 days, 7 to 10 days, 8 to 11 days, 8 to 12 days, 9 to 13 days, 10 to 14 days, 11 to 15 days, 12 to 16 days, 13 to 17 days, 14 to 18 days, 15 to 19 days, or 16 to 20 days.

In another embodiment, the disclosure provides an article of manufacture including a container. The container includes a composition containing an antagonist or antibody as disclosed herein, and a package insert or label indicating that the composition can be used to treat COPD exacerbation and/or symptoms of COPD exacerbations.

In other embodiments, the disclosure provides a kit comprising a composition containing an antagonist or antibody as disclosed herein, and instructions to administer the composition to a subject in need of treatment.

In certain embodiments, antibodies or antagonists for use in the claimed methods comprise a variant Fc region. That is, a non-naturally occurring Fc region, for example an Fc region comprising one or more non-naturally occurring amino acid residues. Also encompassed by the variant Fc regions of the present disclosure are Fc regions which comprise amino acid deletions, additions and/or modifications.

In certain embodiments, an antibody or antagonist for use in the claimed methods has a molecular weight of greater than or equal to about 25 kilodaltons. In other embodiments, an antibody or antagonist for use in the claimed methods has a molecular weight of greater than or equal to about 50, about 75, about 90, about 100, about 110, or about 125 kilodaltons. In other embodiments, an antibody or antagonist has a molecular weight of greater than or equal to about 150 kilodaltons.

The disclosure contemplates the use of antibodies and antagonists having any combination of one or more of the foregoing features. For example, antibodies or antagonists that specifically bind to IL-1R1 and inhibit binding of IL-1α and/or IL-1β and which may have any one or more of the foregoing features can be used in the methods described herein. Similarly, antibodies or antagonists that specifically bind to IL-1α and inhibit binding of IL-1αto IL-1R1 and which may have any one or more of the foregoing features can be used in the methods described herein.

(iv) Methods of Use

In certain embodiments, the antibodies and antagonists used in the claimed methods are useful for treating and/or preventing exacerbation of COPD. In certain embodiments, the antibodies and antagonists used in the claimed methods are useful for increasing lung function during an exacerbation of COPD. In certain embodiments, the antibodies and antagonists used in the claimed methods are useful for decreasing the duration of exacerbations. In certain embodiments, the antibodies and antagonists used in the claimed methods are useful for reducing the frequency of exacerbations. In certain embodiments, the antibodies and antagonists used in the claimed methods are useful for reducing airway inflammation during exacerbations. In certain embodiments, the antibodies and antagonists used in the claimed methods are useful for reducing IL-1α signaling during an exacerbation. In certain embodiments, the antibodies and antagonists used in the claimed methods are useful for reducing IL-1α and IL-1β signaling during an exacerbation. In certain embodiments, exacerbation of COPD is due to an infection of the lung (e.g., viral infection, human rhinovirus-induced airway inflammation, bacterial infection) or air pollultion (e.g., smoke). In certain embodiments, reducing airway inflammation is part of a method of treating COPD exacerbation. In certain embodiments, reducing airway inflammation includes a reduction in inflammatory cell influx into a lung. In certain embodiments, treating COPD exacerbation comprises reducing inflammatory cell influx into a lung. In certain embodiments, the inflammatory cells are neutrophils. In certain embodiments, the inflammatory cells are macrophages. In certain embodiments, the inflammatory cells are lymphocytes. In certain embodiments, the inflammatory cells are mononuclear cells. In certain embodiments, treating COPD exacerbation comprises reducing airway inflammation. In certain embodiments, an antibody for use in the claimed methods has a molecular weight of greater than or equal to about 25 kilodaltons. In certain other embodiments, the antibody used has a molecular weight of greater than or equal to about 50, 60, 75, 100, 110, 125, or 150 kilodaltons. In certain embodiments, the antibody used has a molecular weight of about 150 kilodaltons. Similarly, in certain embodiments, non-antibody antagonists having any of the foregoing ranges of molecular weight are used.

In certain embodiments, the antibodies for use in the claimed methods can be used to treat and/or prevent exacerbation of symptoms of COPD. In certain embodiments, symptoms of an exacerbation of COPD comprise one or more of the following: increased breathlessness, increased cough and sputum production, change in the color and/or thickness of the sputum, wheezing, chest tightness, fever. Exacerbation of COPD represents a change in a patient's baseline, average COPD condition which can be assessed, for example, by assessing lung function.

The Global Initiative for Chronic Obstructive Lung Disease (GOLD) has produced a five-stage classification of COPD severity to guide the therapeutic approach (Executive Summary: Global Strategy for the Diagnosis, Management, and Prevention of COPD (Updated 2009)). In these patients, stage 0 defines the condition characterised by classic clinical symptoms of cough, sputum, and breathlessness without airflow obstruction (e.g., normal spirometry). Stage I defines patients with a forced expiratory volume in one second (FEV1)/forced vital capacity (FVC) of <70%, and an FEV1 of >80% predicted, with or without chronic symptoms that may or may not be aware of disease status. Stage II (FEV1/FVC <70%, FEV1 30-79%) is split into substages IIa (FEV1 50-79%) and IIb (FEV1 30-49%) according to the greater rate of exacerbation experienced by patients in substage IIb, which in turn is inversely related to health status. However, substage IIb is often referred to in the art and herein as stage III. Finally, stage 1V (FEV1/FVC<70% and either FEV1<30% pred, hypoxaemia, or clinical signs of right heart failure) is expected to be associated with the worst health status.

Thus, in certain embodiments, the methods of the disclosure may be used for treating patients with stage I or higher GOLD score COPD, as measured prior to exacerbation. In certain embodiments, the methods of the disclosure may be used for treating patients with stage II or higher GOLD score COPD. In certain embodiments, the methods of the disclosure may be used for treating patients with stage III or higher GOLD score COPD, as assessed prior to exacerbation. In certain embodiments, the methods of the disclosure may be used for treating patients with stage 1V GOLD score COPD, as assessed prior to exacerbation.

In certain embodiments, antibodies for use in the claimed methods can be used to prevent or reduce exacerbation of symptoms of COPD caused by viral infection, bacterial infection, and/or environmental factors. In certain embodiments, the environmental factor is tobacco smoke. In certain embodiments, bacterial infection is associated with LPS. In certain embodiments, viral infection is human rhinovirus (HRV) infection.

In certain embodiments, a method of treating COPD exacerbation in a patient in need thereof, wherein said patient is a patient having COPD exacerbation due to human rhinovirus-induced airway inflammation, comprising administering to said patient an effective amount of a composition comprising an antibody that specifically binds to IL-1R1 and inhibits binding of IL-1R1 to IL-1α is provided. HRV infection causes neutrophil influx with increased inflammatory cytokines. Host inflammatory responses, particularly IL-8, play key roles in pathogenesis of common cold symptoms. In patients with chronic lung diseases this can lead to exacerbation of the symptoms of the underlying respiratory condition. Symptoms of viral infection precede two thirds of COPD exacerbations. 40% of hospitalized acute exacerbation patients have HRV present in nasal and/or sputum samples. Thus, treating patients who have COPD exacerbations due to human rhinovirus-induced airway inflammation represents an important intervention that could significantly reduce the risk of COPD exacerbation and significantly improve the health of patients with COPD. Thus, the present compositions and methods can be useful in treating, reducing and preventing COPD exacerbation induced by HRV or other airway viral infection.

In certain embodiments, an antibody for use in the claimed methods is a human, chimeric or humanized antibody. In certain embodiments, an antibody for use in the claimed methods is an antibody fragment, such as a fragment having a molecular weight of greater than or equal to 25 kilodaltons. In certain embodiments, an antibody or antagonist for use in the claimed methods can specifically bind to human IL-1R1 or IL-1α. In certain embodiments, an antibody or antagonist for use in the claimed methods can specifically bind to IL-1R1 or IL-1α from human and/or from one or more species of non-human primate. In certain embodiments, an antibody for use in the claimed methods does not specifically bind to murine IL-1R1 or IL-1α.

In certain embodiments, the method is part of a therapeutic regimen for treating COPD by managing COPD exacerbation. In certain embodiments, the therapeutic regimen for treating COPD comprises administration of steroids. In certain embodiments, an antibody or antagonist specifically binds to human IL-1R1 or IL-1α with a K_{D} of 50 pM or less when measured by Biacore™. In certain embodiments, an antibody for use in the claimed methods is antibody 6 or 6gl (germlined). In certain embodiments, an antibody or antagonist competes with IL-1Ra for binding to IL-1R1. In certain embodiments, administration is systemic administration. In certain embodiments, the method does not include intranasal administration of said composition. In certain embodiments, the methods comprises administering the antagonist via two route of administration: systemic and local. For example, antagonist is administered systemically, such as intravenously, and intranasally or via other form of local administration to the lung. In certain embodiments, the method comprises administering said composition on a dosing schedule of less than or equal to once daily.

In certain embodiments, COPD symptoms are monitored before, during or after treatment. In certain embodiments, monitoring is continuous. In certain embodiments, monitoring occurs over regular intervals during treatment, such as hourly, daily or weekly. In certain embodiments, monitoring occurs over regular intervals after treatment, such as daily, weekly or monthly. Intervals for monitoring may be readily determined by one of skill in the art based on the severity of the condition. In certain embodiments, COPD symptoms are monitored by pulmonary function tests such as spirometry. In certain embodiments, COPD symptoms are monitored by chest X-ray and/or a computerized tomography (CT) scan. A chest X-ray or CT scan can show emphysema, which is one of the main causes of COPD. In certain embodiments, COPD symptoms are monitored by arterial blood gas analysis. In certain embodiments, COPD symptoms are monitored by sputum examination. In certain embodiments, efficacy of treatment is evaluated using any one or more of the foregoing tests. In certain embodiments, the treatment decreases the severity, duration, or frequency of the exacerbation. In certain embodiments, the patient's condition (e.g., baseline lung function, etc.) returns to the pre-exacerbation baseline levels following treatment.

In certain embodiments, a composition or method of the disclosure is analyzed in a smoke exposed animal model, an animal rhinovirus model or chronic lung disease model that is know to one of ordinary skill in the art. (e.g., Controli et al., Contrib Microbiol. 2007; 14:101-12). In certain embodiments, the animal model is a mouse model (e.g., Bartlett et al., Nat. Med. 2008 February; 14(2):199-204). In certain embodiments, the mouse model is selected from an elastase- and LPS-exposed mouse model (see, Sajjan et al., Am J Physiol Lung Cell Mol. Physiol. 2009 November; 297(5):L931-44). In certain embodiments, any of the cell or animal models set forth in the examples may be used.

In certain embodiments, hospitalization may be required if the symptoms are severe. In certain embodiments, if symptoms are milder, a sufferer may be treated as an outpatient.

In certain embodiments, smoking, hospitalization, lack of a pulmonary rehabilitation program, improper use of an inhaler and poor adherence to a drug therapy program are all associated with more frequent and/or longer duration of episodes of COPD exacerbation. In certain embodiments, the methods of the disclosure may be used for treating patients that display one or more of the following: smoking, hospitalization, lack of a pulmonary rehabilitation program, improper use of an inhaler and poor adherence to a drug therapy program. In certain embodiments, the methods of the disclosure may be used for treating patients with more frequent and/or longer duration of episodes of COPD exacerbation. In certain embodiments, the methods of the disclosure may be used for treating patients at particular risk for COPD exacerbation.

The disclosure also provides a method of antagonising at least one effect of IL-1R1 or IL-1α comprising contacting with or administering an effective amount of one or more antagonists of the present disclosure such that said at least one effect of IL-1R1 or IL-1α is antagonised. Effects of IL-1R1 that may be antagonised by the methods of the disclosure include biological responses mediated by IL-1α and/or IL-1β, and any downstream effects that arise as a consequence of these binding reactions. When multiple antagonists of the disclosure are administered, they may be administered at the same time or a differing times. In certain embodiments, multiple antagonists of the disclosure are used, and the method comprises administering an IL-1R1 antagonist, such as an antibody, and an IL-1alpha antagonist, such as an antibody. Multiple antagonists may be administered via the same route of administration or via differing routes of administration.

For any of the foregoing, the method generally comprises administration of a composition comprising an appropriate dose of the anti-IL-1R1 or IL-1α agent.

The terms “treatment”, “treating”, and the like are used herein to generally mean obtaining a desired pharmacologic and/or physiologic effect by providing a medicament to a subject in need thereof to improve the subject's condition. In certain embodiments, treating may include reducing the frequency and/or severity of exacerbation. In certain embodiments, treating may include treating airway inflammation. In certain embodiments, treating may include preventing or reducing an influx of inflammatory cells, such as neutrophils, into the lung. “Treatment” as used herein includes: (a) inhibiting the exacerbation (e.g., arresting its development so that symptoms do not worsen); or (b) relieving the disease or condition (e.g., causing regression of the disease or condition, providing improvement in one or more symptoms, decreasing duration of exacerbation, decreasing frequency of exacerbation). Improvements in any conditions can be readily assessed according to standard methods and techniques known in the art. In certain embodiments, following effective treatment, the patient's condition returns to their pre-exacerbation baseline condition. In certain embodiments, prior to exacerbation, the patient has moderate or severe COPD (e.g., COPD classified as GOLD stage III or GOLD stage 1V).

By the term “therapeutically effective dose” or “effective amount” is meant a dose that produces the desired effect for which it is administered. The exact dose will depend on the purpose of the treatment, and will be ascertainable by one skilled in the art using known techniques (see, e.g., Lloyd (1999) The Art, Science and Technology of Pharmaceutical Compounding).

The disclosure contemplates methods in which one or more of any of the foregoing or following aspects and/or embodiments of the disclosure are combined. For example, any antibody or antagonist (any composition that antagonizes IL-1R1 or IL-1α) can be used in any of the methods described herein. Moreover, any antibody or antagonist describes herein may be used alone or in combination, such as in combination with another antibody or antagonist of the disclosure.

(v) Pharmaceutical Compostions

Accordingly, further aspects of the disclosure provide the use of an antibody or antagonist to treat COPD exacerbation, as described herein. Antibodies and antagonists can be administered as compositions, for example pharmaceutical compositions comprising an antibody or antagonist. In certain embodiments, the antibody or antagonist is produced recombinantly, such as by expressing nucleic acid encoding the antibody or antagonist in a host cell. Compositions can be formulated in a pharmaceutically acceptable excipient. In certain embodiments, the composition is pyrogen free or substantially pyrogen free.

A pharmaceutically acceptable excipient may be a compound or a combination of compounds entering into a pharmaceutical composition not provoking secondary reactions and which allows, for example, facilitation of the administration of the active compound(s), an increase in its lifespan and/or in its efficacy in the body, an increase in its solubility in solution or else an improvement in its conservation. These pharmaceutically acceptable excipients are well known and will be adapted by the person skilled in the art as a function of the nature and of the mode of administration of the active compound(s) chosen.

Antibodies and antagonists of the present disclosure will usually be administered in the form of a pharmaceutical composition, which may comprise at least one component in addition to the antagonist. Thus pharmaceutical compositions according to the present disclosure, and for use in accordance with the present disclosure, may comprise, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration. In certain embodiments, the composition is administered systemically, such as by intravenous, intra-peritoneal, intra-muscular, or subcutaneous injection. In certain embodiments, the composition is administered orally. In certain embodiments, the method specifically does not include administration of the composition directly to the lungs, for example by inhalation, pulmonary lavage, or intra-nasal delivery. In other embodiments, the same or different antibodies/antagonists are administrated via the same or differing routes of andministration. For example, an antibody may be administrated systemically, and the same or a different antagonist may be administered systemically or locally.

Liquid pharmaceutical compositions generally comprise a liquid carrier, such as water, petroleum, animal or vegetable oils, mineral oil or synthetic oil. Physiological saline solution, dextrose or other saccharide solution or glycols, such as ethylene glycol, propylene glycol or polyethylene glycol may be used or included.

For intra-venous injection, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles, such as Sodium Chloride Injection, Ringer's Injection, Lactated Ringer's Injection. Preservatives, stabilizers, buffers, antioxidants and/or other additives may be employed as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g. Zn-protein complexes); and/or non-ionic surfactants, such as TWEEN™, PLURONICS™ or polyethylene glycol (PEG).

Antagonists and antibodies of the present disclosure may be formulated in liquid, semi-solid or solid forms depending on the physicochemical properties of the molecule and the route of delivery. Formulations may include excipients, or combinations of excipients, for example: sugars, amino acids and surfactants. Liquid formulations may include a wide range of antibody concentrations and pH. Solid formulations may be produced by lyophilisation, spray drying, or drying by supercritical fluid technology, for example.

In certain embodiments, compositions of the disclosure, including pharmaceutical compositions, are non-pyrogenic. In other words, in certain embodiments, the compositions are substantially pyrogen free. In one embodiment, the formulations are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with antibodies, even trace amounts of harmful and dangerous endotoxin must be removed. In certain specific embodiments, the endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

In certain embodiments, the composition is administered by intravenous infusion. In certain embodiments, infusion is over a period of at least 10, at least 15, at least 20, or at least 30 minutes. In other embodiments, infusion is over a period of at least 60, 90, or 120 minutes. Regardless of the infusion period, the disclosure contemplates that each infusion is part of an overall treatment plan where antibody or antagonist is administered according to a regular schedule (e.g., once per day, weekly, monthly, etc.). Similarly, regardless of route of administration, the disclosure contemplates that each dose is part of an overall treatment plan where antibody or antagonist is administered according to a regular schedule (e.g., once per day, weekly, monthly, etc.).

In certain embodiments, a composition of the disclosure (e.g., an anti-IL-1R1 antibody, an anti-IL-1α antibody, an anti-IL-1R1 antagonist) may be used as part of a combination therapy or therapeutic regimen for treating COPD exacerbation. Combination treatments may be used to provide additive or synergistic effects, particularly the combination of an anti-IL-1R1 or IL-1α, antagonist with one or more other drugs. When a therapeutic regimen involves administration of multiple compounds (e.g., drugs, biological agents), such compounds may, for example, be administered concurrently or sequentially or as a combined preparation. In certain embodiments, the therapeutic regimen includes steroid therapy.

In certain embodiments, compositions of the disclosure may be used as part of a therapeutic regimen with one or more available treatments for COPD.

Compositions according to the present disclosure may be provided as sole therapy or in combination or addition with one or more other agents of the disclosure and/or with one or more of the following agents:

a glucocorticoid, such as flunisolide, triamcinolone acetonide, beclomethasone dipropionate, budesonide, fluticasone propionate, ciclesonide, and/or mometasone furoate;

an antibacterial agent, e.g. a penicillin derivative, a tetracycline, a macrolide, a beta-lactam, a fluoroquinolone, metronidazole and/or an inhaled aminoglycoside; and/or an antiviral agent, e.g. acyclovir, famciclovir, valaciclovir, ganciclovir, cidofovir; amantadine, rimantadine; ribavirin; zanamavir and/or oseltamavir; a protease inhibitor, such as indinavir, nelfinavir, ritonavir and/or saquinavir; a nucleoside reverse transcriptase inhibitor, such as didanosine, lamivudine, stavudine, zalcitabine, zidovudine; a non-nucleoside reverse transcriptase inhibitor, such as nevirapine, efavirenz.

Combination treatment may include antibiotics. Approximately 50% of acute exacerbations are due primarily to the bacteria Streptococcus pneumoniae (causing pneumonia), Haemophilus influenzae (causing flu), and Moraxella catarrhalis (causing pneumonia). Numerous antibiotics may effectively treat these infections.

Combination treatment may include respiratory stimulants. Corticosteroids may be beneficial in acute exacerbations of COPD. Steroids may be given intravenously. Bronchodilator dosages may be increased during acute exacerbations to decrease acute bronchospasm. Theophylline may be used during acute exacerbations of COPD.

In certain embodiments, oxygen requirements may increase and supplemental oxygen may be provided.

Patients with acute exacerbations of COPD may be at risk of developing respiratory failure. Respiratory failure occurs when respiratory demand exceeds the ability of the respiratory system to respond. In certain embodiments, combination may include mechanical ventilation.

Mechanical ventilation is a means by which air is pushed into a patient's lungs by the ventilator instead of the patient using his respiratory muscles to draw in air. Mechanical ventilation therefore reduces or eliminates the patient's work of breathing, and the patient continues to receive air into his lungs and passively exhale without any work. There are two commonly used methods for mechanical ventilation in COPD: noninvasive and invasive.

During invasive ventilation an endotracheal tube, a small-diameter plastic tube, is placed into the trachea and then connected to a ventilator, which pushes air into the lungs. Invasive ventilation may be administered to patients who are unconscious or heavily sedated, and it is more effective than noninvasive ventilation.

Noninvasive ventilation may be used in a conscious, cooperative patient. In this method, oxygen is delivered through a mask that forms a seal around the nose or mouth and nose.

In certain embodiments, combination treatment may include pneumonia and/or annual flu vaccines.

In accordance with the present disclosure, compositions provided may be administered to mammals, such as human patients. Administration is normally in a “effective amount”, this being sufficient to show benefit to a patient. Such benefit may be at least amelioration of at least one symptom. Exemplary symptoms include airway inflammation, neutrifil influx into lung, decreased lung capacity.

The actual amount administered, and rate and time-course of administration, will depend on the nature and severity of what is being treated, the particular mammal being treated, the clinical condition of the individual patient, the cause of the disorder, the site of delivery of the composition, the type of antagonist, the method of administration, the scheduling of administration and other factors known to medical practitioners. Prescription of treatment, e.g. decisions on dosage etc, is within the responsibility of general practitioners and other medical doctors and may depend on the severity of the symptoms and/or progression of a disease being treated. Appropriate doses of antibody are well known in the art [33, 34]. Specific dosages indicated herein or in the Physician's Desk Reference (2003) as appropriate for the type of medicament being administered may be used. A therapeutically effective amount or suitable dose can be determined by comparing its in vitro activity and in vivo activity in an animal model. Methods for extrapolation of effective dosages in mice and other test animals to humans are known. The precise dose will depend upon the precise nature of the antibody (e.g. whole antibody, fragment or diabody), patient condition, dosing schedule. A typical antibody dose will be in the range 100 μg to 1 g for systemic applications. In certain embodiments, an initial higher loading dose, followed by one or more lower doses, may be administered. Typically, the antibody will be a whole antibody, e.g. the IgG1 isotype, IgG2 isotype, IgG3 isotype or IgG4 isotype. This is a dose for a single treatment of an adult patient, which may be proportionally adjusted for children and infants, and also adjusted for other antibody formats in proportion to molecular weight. Treatments may be repeated at daily, twice-weekly, weekly or monthly intervals, at the discretion of the physician. In certain embodiments, treatments may be every two to four weeks for subcutaneous administration and every four to eight weeks for intra-venous administration. In certain embodiments, compositions of the disclosure require periodic dosing for the remainder of the subject's life.

In certain embodiments, compositions of the disclosure are administered systemically. In certain embodiments, compositions of the disclosure are administered by i.v. In certain embodiments, compositions of the disclosure are not effectively delivered by inhallation. In certain embodiments, compositions of the disclosure are not effectively delivered non-systemically. In certain embodiments, compositions of the disclosure require continuous dosing. In certain embodiments, compositions of the disclosure require continuous dosing for period of a day, 2, 3, 4, 5, 6 or 7 days. In certain embodiments, compositions of the disclosure require continuous dosing for period of a week, 2, 3, 4, 5, or 6 weeks. In certain embodiments, compositions of the disclosure require continuous dosing for period of a month, 2, 3, 4, 5, 6, 7, 8, 9, 10, 11 or 12 months. In certain embodiments, compositions of the disclosure require continuous dosing for the remainder of the subject's life.

(vi) Preparation of Antibodies and Antagonists

In certain aspects, the present disclosure provides methods in which the effective agent is an antibody that specifically binds to IL-1R1. In certain aspects, the present disclosure provides methods in which the effective agent is an antibody that specifically binds to IL-1α. Exemplary antibodies include murine, chimeric, humanized, and human antibodies, as well as antigen binding fragments. Suitable antibodies can be prepared using methods well known in the art. For example, antibodies can be generated recombinantly, made using phage display, produced using hybridoma technology, etc. Non-limiting examples of techniques are described briefly below.

In general, for the preparation of monoclonal antibodies or their functional fragments, especially of murine origin, it is possible to refer to techniques which are described in particular in the manual “Antibodies” [35] or to the technique of preparation from hybridomas described by Köhler and Milstein [36].

Monoclonal antibodies can be obtained, for example, from a cell obtained from an animal immunized against IL-1R1 or IL-1α, or one of its fragments containing the epitope recognized by said monoclonal antibodies. Suitable fragments and peptides or polypeptides comprising them may be used to immunise animals to generate antibodies against IL-1R1 or IL-1α. Said IL-1R1 or IL-1α, or one of its fragments, can especially be produced according to the usual working methods, by genetic recombination starting with a nucleic acid sequence contained in the cDNA sequence coding for IL-1R1 or IL-1α or fragment thereof, by peptide synthesis starting from a sequence of amino acids comprised in the peptide sequence of the IL-1R1 or IL-1α and/or fragment thereof.

The monoclonal antibodies can, for example, be purified on an affinity column on which IL-1R1 or IL-1α or one of its fragments containing the epitope recognized by said monoclonal antibodies, has previously been immobilized. More particularly, the monoclonal antibodies can be purified by chromatography on protein A and/or G, followed or not followed by ion-exchange chromatography aimed at eliminating the residual protein contaminants as well as the DNA and the lipopolysaccaride (LPS), in itself, followed or not followed by exclusion chromatography on Sepharose™ gel in order to eliminate the potential aggregates due to the presence of dimers or of other multimers. In one embodiment, the whole of these techniques can be used simultaneously or successively.

It is possible to take monoclonal and other antibodies and use techniques of recombinant DNA technology to produce other antibodies or chimeric molecules that bind the target antigen. Such techniques may involve introducing DNA encoding the immunoglobulin variable region, or the CDRs, of an antibody to the constant regions, or constant regions plus framework regions, of a different immunoglobulin. See, for instance, EP-A-184187, GB 2188638A or EP-A-239400, and a large body of subsequent literature. A hybridoma or other cell producing an antibody may be subject to genetic mutation or other changes, which may or may not alter the binding specificity of antibodies produced.

Further techniques available in the art of antibody engineering have made it possible to isolate human and humanised antibodies. For example, human hybridomas can be made as described by Kontermann & Dubel [37]. Phage display, another established technique for generating antagonists has been described in detail in many publications, such as Kontermann & Dubel [37] and WO92/01047 (discussed further below), and US patents U.S. Pat. No. 5,969,108, U.S. Pat. No. 5,565,332, U.S. Pat. No. 5,733,743, U.S. Pat. No. 5,858,657, U.S. Pat. No. 5,871,907, U.S. Pat. No. 5,872,215, U.S. Pat. No. 5,885,793, U.S. Pat. No. 5,962,255, U.S. Pat. No. 6,140,471, U.S. Pat. No. 6,172,197, U.S. Pat. No. 6,225,447, U.S. Pat. No. 6,291,650, U.S. Pat. No. 6,492,160 and U.S. Pat. No. 6,521,404.

Transgenic mice in which the mouse antibody genes are inactivated and functionally replaced with human antibody genes while leaving intact other components of the mouse immune system, can be used for isolating human antibodies [38]. Humanised antibodies can be produced using techniques known in the art such as those disclosed in, for example, WO91/09967, U.S. Pat. No. 5,585,089, EP592106, U.S. Pat. No. 5,565,332 and WO93/17105. Further, WO2004/006955 describes methods for humanising antibodies, based on selecting variable region framework sequences from human antibody genes by comparing canonical CDR structure types for CDR sequences of the variable region of a non-human antibody to canonical CDR structure types for corresponding CDRs from a library of human antibody sequences, e.g. germline antibody gene segments. Human antibody variable regions having similar canonical CDR structure types to the non-human CDRs form a subset of member human antibody sequences from which to select human framework sequences. The subset members may be further ranked by amino acid similarity between the human and the non-human CDR sequences. In the method of WO2004/006955, top ranking human sequences are selected to provide the framework sequences for constructing a chimeric antibody that functionally replaces human CDR sequences with the non-human CDR counterparts using the selected subset member human frameworks, thereby providing a humanized antibody of high affinity and low immunogenicity without need for comparing framework sequences between the non-human and human antibodies. Chimeric antibodies made according to the method are also disclosed.

Synthetic antibody molecules may be created by expression from genes generated by means of oligonucleotides synthesized and assembled within suitable expression vectors, for example as described by Knappik et al. [39] or Krebs et al. [40].

Note that regardless of how an antibody of interest is initially identified or made, any such antibody can be subsequently produced using recombinant techniques. For example, a nucleic acid sequence encoding the antibody may be expressed in a host cell. Such methods include expressing nucleic acid sequence encoding the heavy chain and light chain from separate vectors, as well as expressing the nucleic acid sequences from the same vector. These and other techniques using a variety of cell types are well known in the art.

Suitable antibodies can be tested in one or more assays. For example, antibodies can be tested in any of the assays provided in the examples to confirm that they possess similar functional properties as these representative antibodies. Additionally or alternatively, antibodies can be tested to assess whether they bind to the same or substantially the same epitope as any of those antibodies. Binding assays to confirm that antibodies specifically bind target antigen from one or more desired species can also be performed. Further, neutralization capacity (e.g., the ability of an anti-IL-1R1 antibody to prevent binding of IL-1R1 to IL-1alpha and/or beta can be tested.

In the case of non-antibody antagonists, such antagonists can be prepared using methods known in the art. For example, protein antagonists can be prepared using recombinant technology or synthetically. An exemplary protein antagonist is KINERET, a commercially available form of IL-1Ra.

### EXEMPLIFICATION

The disclosure now being generally described, it will be more readily understood by reference to the following examples, which are included merely for purposes of illustration of certain aspects and embodiments of the present disclosure, and are not intended to limit the disclosure. For example, the particular constructs and experimental design disclosed herein represent exemplary tools and methods for validating proper function.

### Example 1

### IL-1R1 Blockade Inhibits the Effects of IL-1beta in Vitro and in Vivo

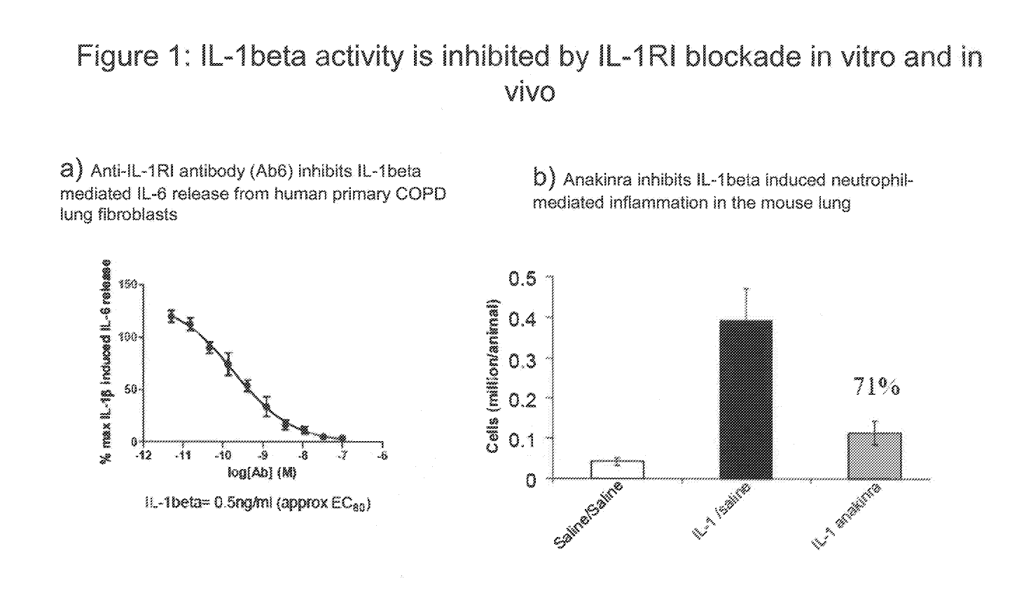

Some of the tools used in this and further examples are antibody 6 (a human antibody that binds specifically to IL-1R1; sequence provided herein) and anakinra (also known as KINERET). Antibody 6 completely inhibited IL-1beta induced IL-6 in primary human COPD fibroblasts (FIG. 1A) and anakinra inhibited by 71% the ability of IL-1beta, when instilled intratracheally into mice, to increase neutrophils recovered in BAL 4 hours later (FIG. 1B). This is consistent with the literature for anakinra and for other anti-IL-1R1 antibodies, such as the anti mouse IL-1R1 antibdy 35F5, which have been shown to inhibit IL-1beta mediated effects at IL-1R1. As further described in the examples, the present disclosure revealed additional and surprising effects on IL-1alpha mediated activity, thus implicating IL-1alpha in COPD for the first time.

To examine the effect of IL-1R1 on IL-1beta in COPD tissue, IL-6 levels were examined in primary COPD lung fibroblasts treated with an IL-1R1 antagonist. The IL-1R1 antagonistic antibody 6 (a human antibody that specifically binds human IL-1R1; germlined version used in this experiment) inhibited IL-1beta induced IL-6 release in COPD lung fibroblasts (FIG. 1A). The IL-1beta treatment concentration was 0.5 ng/ml (approximately EC₈₀).

As noted above, the effect of IL-1R1 antagonist treatment on an IL-1beta-induced neutrophil mediated inflammation in the mouse lung was also examined (FIG. 1B). Anakinra (KINERET™) was dosed subcutaneously one hour before a treatment of IL-1beta of 5 ng/50 μl. After four hours, cell counts were obtained by BAL. Anakinra reduced the cell count by 71% compared to control IL-1 treated animals.

### In Vitro Methods:

COPD fibroblasts were generated as a bi-product of the generation of endothelial cells from COPD lung tissue from severe COPD patients receiving lung transplantation. At the time of tissue removal, patients' disease was stable and not exacerbating.

Tissue culture flasks were coated with gelatin (0.2% in distilled water) after sterile filtering and were rinsed with cell media before use.

Tissue was dissected from pleura and chopped using a mezzaluna in RPMI+ (RPMI+ was RPMI media+10% FCS, 1% penicillin/streptomycin/amphotericin B solution) media. Chopped tissue (when fine enough to be sucked easily into a standard pasteur pipette) was washed on a 40 micron filter to remove debris and red cells. Cells were removed from the filter using a sterile instrument and resuspended for digestion in RPMI, 0.1% BSA and 0.2% collagenase type II. The tissue was incubated on a roller for 2 hrs at room temperature. The tube was shaken gently occasionally to prevent the tissue from clumping and settling. After 2 hrs, the suspension was gently agitated and then filtered through a large mesh strainer and then through 100 micron filters. The filtrate was then spun at 1200 rpm for 5 minutes at room temperature. The cell pellet was then washed in RPMI+ and the spin and the wash were repeated. The cells were then resuspended in endothelial culture media (EGM-2-MV BulletKit, CLonetics #CC3202) and plated into gelatin coated flasks. Cells were plated at about 2e7 cells per T225 flask. The next day, media was flushed across the cells, cells were passaged using cell dissociation fluid when they approached confluence. At this point endothelial cells were enriched using CD31 Dynabeads, cells which were negative for association to beads were mostly fibroblasts and could be counted and used for COPD fibroblast assays.

From this point cells were cultured in DMEM supplemented with 10% fetal calf serum (FCS). Fibroblast cells were plated at 1e5 cells per well in 96 well flat bottomed polystyrene plates and were incubated overnight at 37° C. to allow adherence. Antibody or medium alone was preincubated with cells in duplicate wells for 30 minutes prior to addition of IL-1beta (R&D Systems 201-LB/CF) at an IL-1beta concentration of 0.5 ng/ml final assay concentration. Final volume in each well was 200 ul. The plate was incubated at 37° C. 5% CO₂ for 24 hours. The plate was spun briefly before supernatants were removed for analysis of IL-6 levels using R&D Systems ELISA (DY206).

In Vivo Methods:

Mice were adult Balb/c females. Anakinra was dosed subcutaneously one hour before IL-1beta was administered intratracheally to the mouse lung using a dose of 5 ng in 50 ul. After 4 hours, the lungs of the mouse were lavaged, essentially as for the acute smoke model (example 2), and total cells and differential cell counts performed.

### Example 2

### IL-1R1 Antagonists Inhibit Cell Influx in an Acute Tobacco Smoke Model of Lung Inflammation

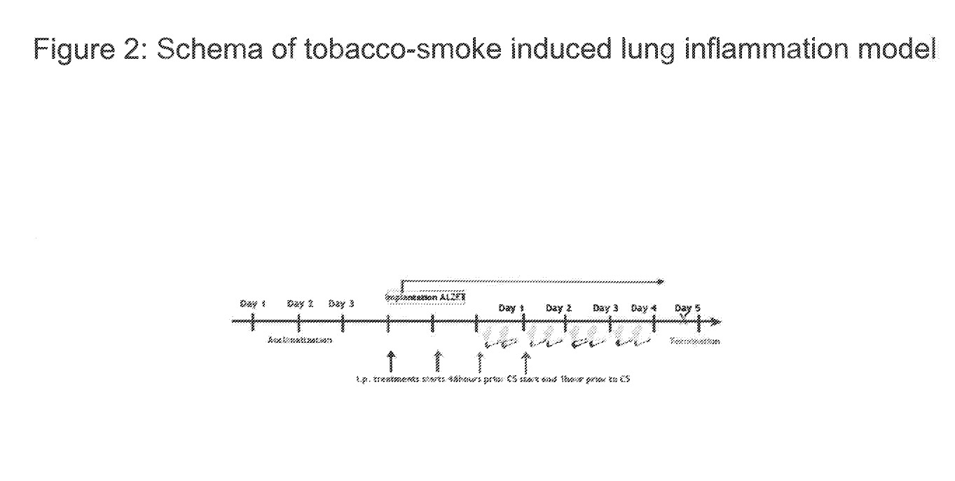

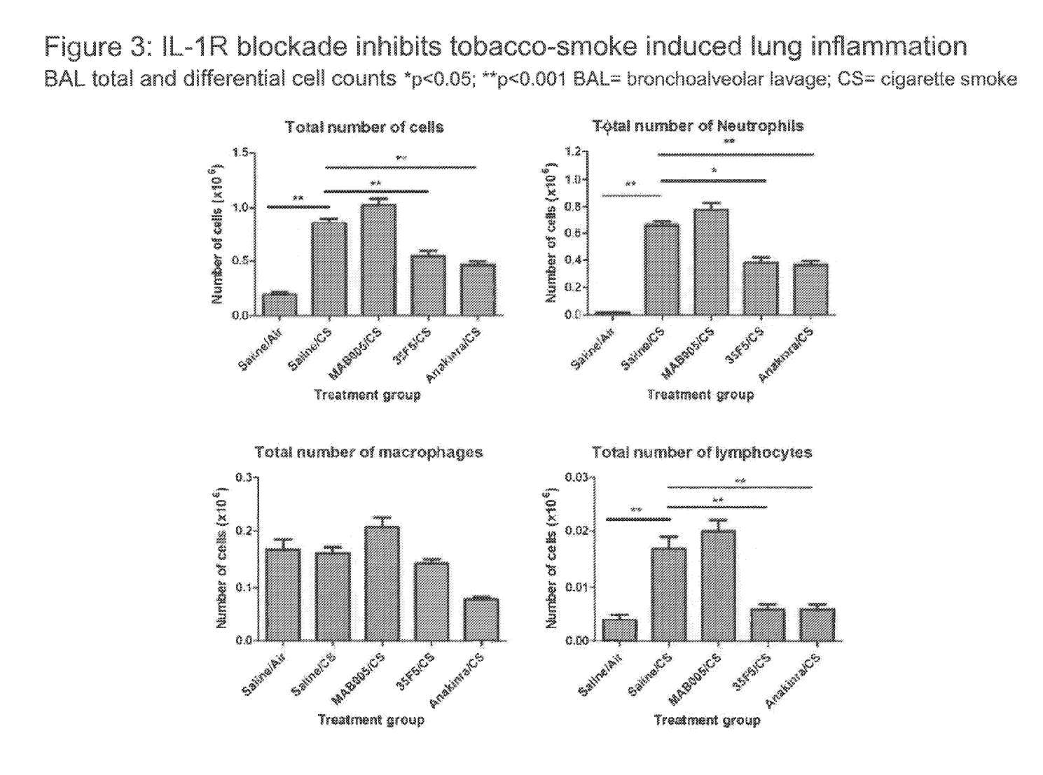

The antibody 35F5 is a monoclonal antibody which binds to mouse IL-1R1 and prevents the binding of both IL-1beta and IL-1alpha to the receptor IL-1R1Anakinra antagonises the effects of both IL-1beta and IL-1alpha. Interleukin 1 shows strong disease association with stable disease and smoking-induced alterations in inflammatory processes in humans. The data shown in this example confirms that inhibition of IL-1R1 by 35F5 decreases inflammation in an acute model of murine lung inflammation, induced by a stimulus relevant to COPD such as smoke. This is consistent with previous observations in the public domain, and with other studies using IL-1Ra (anakinra; IL-1 receptor antagonist). In this mouse model, cigarette smoke causes significant increases in neutrophil BAL cell numbers after 4 days of smoke inhalation. To investigate the effects of IL-1R1 pathway inhibition on the acute inflammatory response to cigarette smoke inhalation, Balb/c mice were exposed to cigarette smoke twice daily (for 50 minutes) for 5 days and dosed intraperitoneally once daily with either 35F5, isotype control rat IgG1 (MAB005), or saline, starting 48 hours before the first smoke exposure and continuing for 4 days. On Day 5, animals were terminated and BAL was performed. An additional treatment arm was included in which animals were exposed to cigarette smoke as above but dosed sub-cutaneously (SC) continuously with anakinra using infusion pumps (ALZET) starting dosing 48 hours prior to the first smoke exposure. Both 35F5 and anakinra administered by ALZET significantly inhibited tobacco smoke-induced acute inflammatory cell infiltration in BAL of mice, whereas the isotype control antibody (MAB005) had no effect. 35F5 significantly reduced smoke-induced increases in total cells (p<0.001), neutrophils (p<0.01), and lymphocytes (p<0.001). In this study, there was no significant increase in macrophages in BAL in response to smoke exposure. A summary of the effect of smoke exposure, and inhibition by IL-1R1 antagonists, on lung inflammatory cells is provided in FIG. 3. The protocol for the study is shown in FIG. 2 and described in the methods. Implantation of the osmotic pump for ALZET treatments was performed between acclimatization and treatment in order to allow recovery before treatment. 35F5 is a commercially available rodent antibody sold by BD Biosciences/BD Pharmingen. The MAB005 isotype control is available from R&D Systems.

### Methods:

Adult Balb/c female mice were used for both studies. The antibody 35F5 was sourced from BD Bioscience (San Diego) (Purified NA/LE Rat anti-mouse CD121a catalogue number 624094) and was a rat IgG1 monoclonal antibody specific to IL-1R1. It contained very low levels of endotoxin (<0.01 ng/ug endotoxin) and no preservatives. The rat isotype control was sourced from R&D Systems (catalogue number MAB005, batch CAN070905A) and contained low endotoxin levels (<0.1EU/ug). Anakinra was obtained from a pharmacy-Kineret DB00026 (BTD00060; BIOD00060) Lot number1004729(004699) exp 072009 (Amgen). Kentucky research grade cigarettes IR3F with removed filter were used (Tobacco and Health Research Institute, University of Kentucky). Osmotic pumps used to continuously administer anakinra in some mice were ALZET model 2001, nominal performance (at 37° C.) 0.93 ul/hr, 7 days duration, 0.23 ml reservoir volume. Pumps were filled with anakinra which had been brought to room temperature (protected from light). Stock of 150 mg/ml was diluted in isotonic saline in order to provide a dose of 48 mg/kg/day and the pumps were filled in sterile conditions following the manufacturer's instructions.

Animals were received at least 7 days prior to experimental start and were acclimatised to the exposure box for increased periods of time connected to the smoking machine without receiving smoke, and were kept in a facility with a 12 hr light/dark cycle at 21±2° C. and with 55±15% humidity. They were fed and watered ad libitum with standard chow and tap water. Prior to study start, animals were randomised into groups. Those animals having osmotic pump implantation were weighed and anaesthetised with isofluorane mixture (N₂O, O₂ 1.4:1.2 and 3% isoflurane) and under narcosis, the region scapulae sinister was shaved and cleaned before a small dorso-ventral skin incision was made 5 mm behind the margus caudalis scapulae. The incision area was soaked in a sanitising fluid (Marcain 50 mg/ml) before a pocket was opened up in the subcutaneous tissue with scissors. A filled pump was inserted into the pocket, delivery portal first, to minimize the interaction with the incision. The incision was closed under sterile conditions with sutures and the mouse observed until recovery. The cigarette smoke (CS) sessions began no less than 48 hours after this procedure.

Antibodies (or anakinra in i.p. anakinra groups) were administered intraperitoneally (i.p.) (4 injections as per individual study schedules) in <200 ul volume to no more than 10 ml/kg body weight. Antibodies (or anakinra in i.p. anakinra groups) were dosed at a nominal concentration of 15 mg/kg.

48 hours after osmotic pump implantation or 1 hr after i.p. antibody administration the mice receive their first smoking session. Mice were positioned randomly in a whole-body exposure box at every smoke exposure session and exposed to smoke for 50 minutes twice daily on days 1-4. Smoke for 50 minutes equates to 10 cigarettes; the smoke machine alternates air and ‘puffs’ of smoke. The control group received the same procedure but with air instead of smoke. The mice were terminated on day 5 (16 hours after the final smoke exposure) by administration of pentobarbital. After exposure of the trachea, the lungs were lavaged with room temperature PBS (w/o Mg and Ca) at 23 cm of hydrostatic pressure (2 min in and 1 min out and repeated). Cells were centifugated-supernatants could be analysed for mediators and the cells were analysed for total cells and for differential cell counting using an automated counter such as Sysmex XT-1800i Vet. The significance of differences between groups was calculated using Student t-test, with one-tailed distribution and two-sample unequal variance as a minimum of significance (one sided Students t-test, unequal variances). Limits for p-values are p≦0.05.

### Example 3

### IL-1 Alpha Plays a Key Role in Inflammation Driven by Tobacco Smoke in an Acute Mouse Model

There is no study describing the inhibition of IL-1alpha in a smoke induced inflammation model. Both IL-1alpha and IL-1beta induce equivalent activation of IL-1R1 at similar concentrations in vitro in simple activity assays, and therefore, we postulated that IL-1alpha and IL-1beta if present in disease could both activate IL-1R1. However, the literature did not yet describe any involvement of IL-1alpha in disease. Here we demonstrate that IL-1alpha plays a critical role in acute smoke induced inflammation.

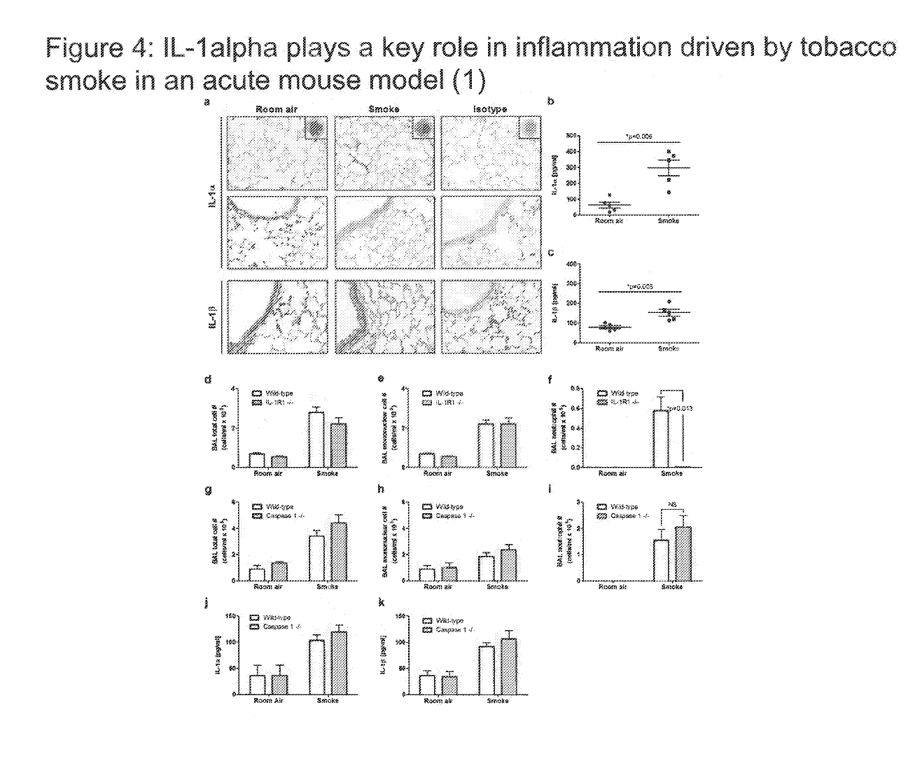

First we demonstrated that both IL-1alpha and IL-1beta were present in the lungs of smoke-exposed mice. Expression of IL-1α in room air control mice was mainly confined to macrophages within the alveolar spaces and, occasionally, to intra-epithelial cells within the bronchiolar mucosa, and a low grade staining was noted on the occasional bronchiolar epithelial cell and epithelial secretory cell (FIG. 4A). In the smoke-exposed mice, a marked IL-1α expression on the expanded alveolar macrophage population was the key histological phenotype; although, IL-1α staining was also noted on the occasional hyperplastic bronchiolar epithelial cell. Of note, infiltrating cells within the bronchiolar and vascular adventitia compartments were negative.

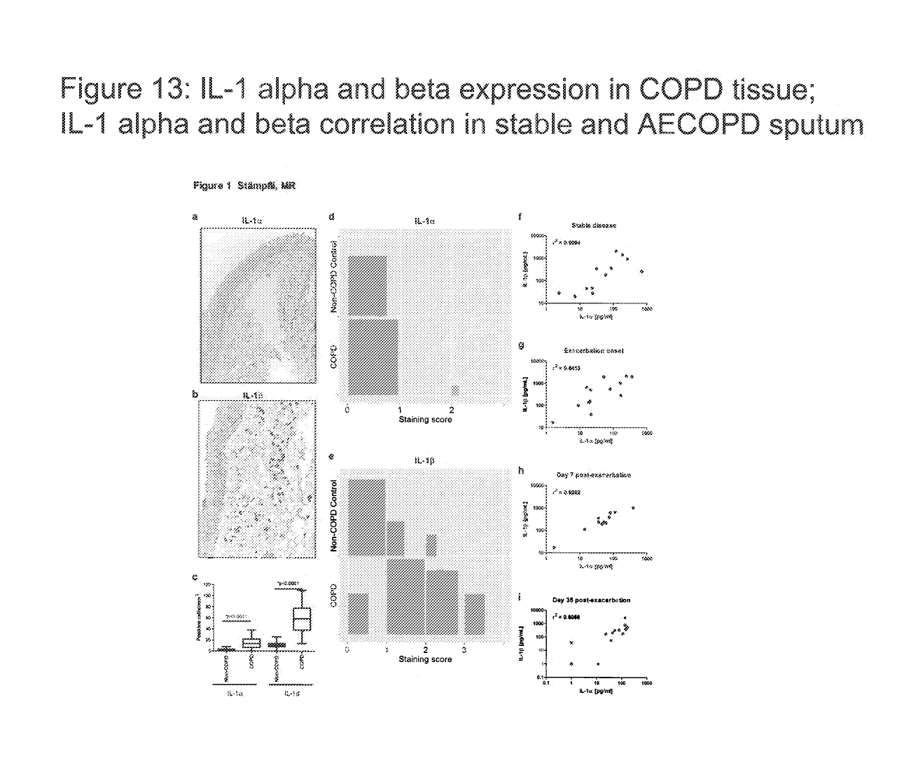

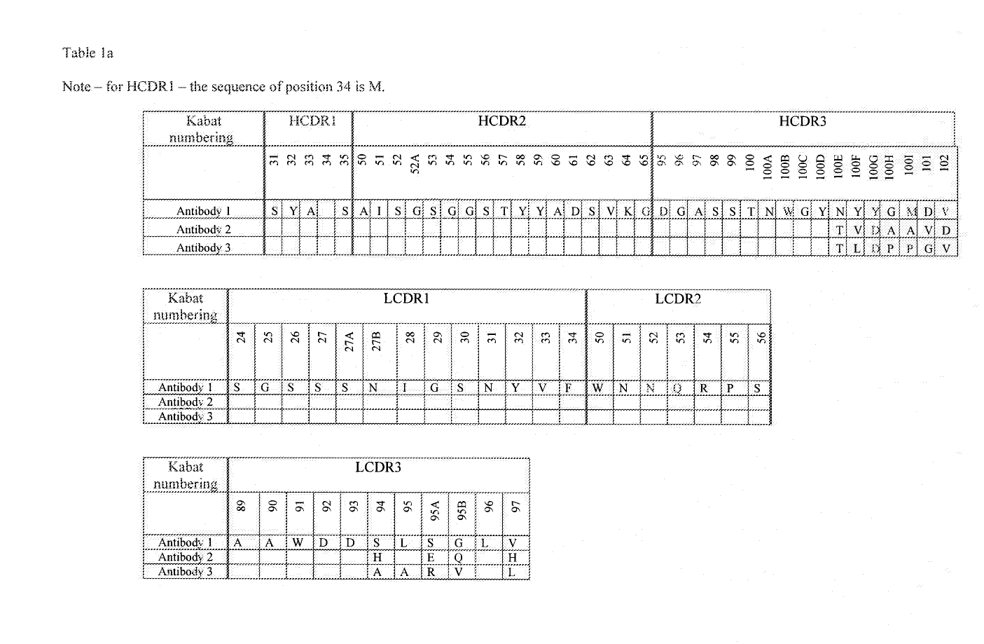

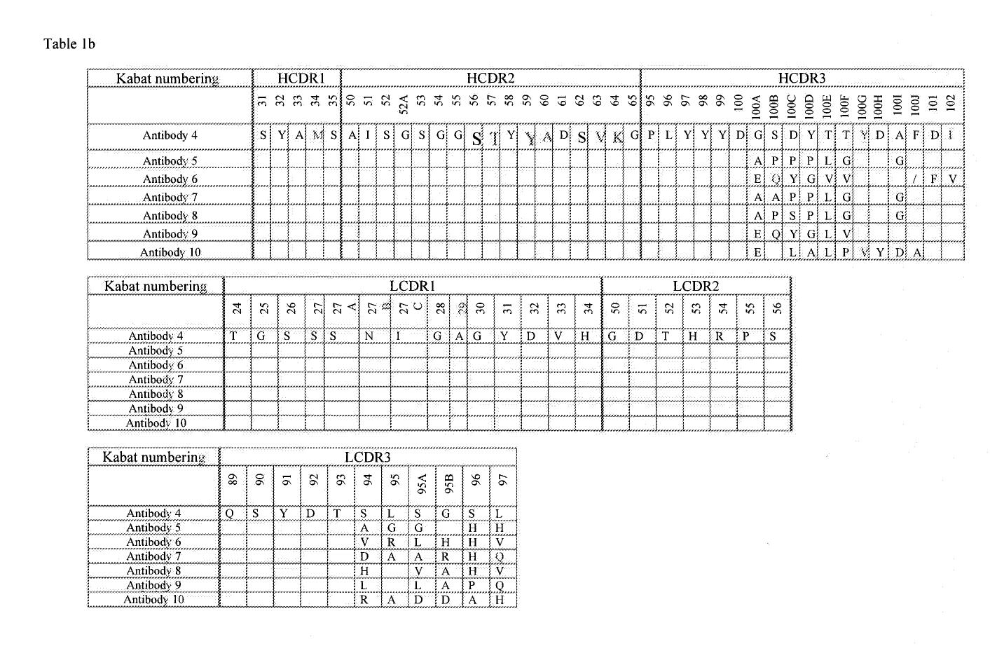

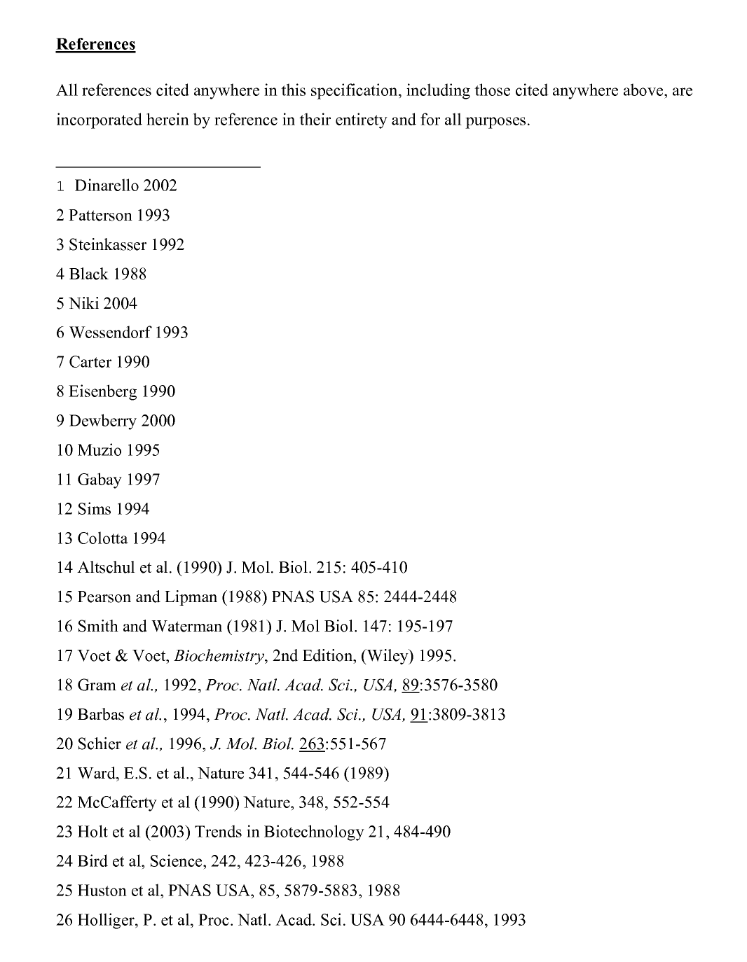

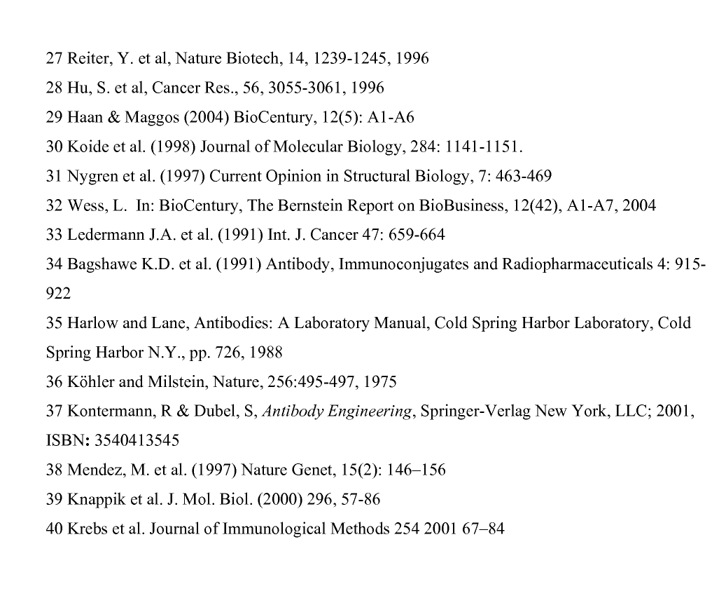

In contrast to the IL-1α expression pattern, widespread tissue expression of IL-1β was observed in room air and smoke-exposed mice (FIG. 4A). In room air controls, a variable expression was noted on the alveolar macrophage population. In addition, there was expression on alveolar type I (ATI) and ATII cells, especially in the terminal alveolar buds of ATII cells, and on the occasional hypertrophic ATII cell. In smoke-exposed animals, a marked staining in the expanded alveolar macrophage population was observed. Moreover, increased expression was observed in both the ATI and ATII cells, especially the hypertrophic forms. Widespread and marked expression of IL-1β was also observed on the bronchiolar epithelium. This was particularly evident on hypertrophic cells, and epithelial secretory cells. As can be seen by comparison to Example 12 and FIGS. 13A and B, tissue expression of IL-1α and β in smoke-exposed mice involves a similar population of both inflammatory infiltrate and resident cells to that seen in COPD patients.

Given the similarities between the expression profile of IL-1α and β in samples from COPD patients and in the above mouse model, we used this experimental model as a platform to examine the functional importance of IL-1α and IL-1β to cigarette smoke-induced inflammation and viral exacerbation. The foregoing is expected to mimic COPD and COPD exacerbation. We observed increased levels of total IL-1α and IL-1β in the lungs of smoke-exposed animals compared to controls (FIGS. 4B and 4C, respectively).

To assess the role of neutrophilic inflammation in our model, IL-1R1 deficient and wild-type mice were exposed to cigarette smoke. Neutrophilia was completely attenuated in the bronchoalveolar lavage (BAL) of IL-1R1 deficient animals compared to wild-type controls (FIG. 4F). An IL-1R1 deficiency did not impact total or mononuclear cell numbers in the BAL of smoke-exposed mice (FIGS. 4D and 4E, respectively). While the expression of neutrophil recruiting chemokines, CXCL-1, -2, and -5, were increased following smoke-exposure of wild-type mice, IL-1R1 deficiency significantly decreased this induction.

Given that caspase-1 cleaves pro-IL-1β into its bio-active form and that this process has been shown to contribute to cigarette smoke-induced neutrophilic inflammation, we exposed caspase-1 deficient mice to cigarette smoke. Caspase-1 deficiency did not significantly alter smoke-induced neutrophilia in the BAL (FIG. 41). Similarly, the total and mononuclear cell numbers of the BAL were not decreased in smoke-exposed caspase-1 deficient mice compared to wild-type controls (FIGS. 4G and H, respectively). Interestingly, we observed similar levels of total IL-1α and IL-1β protein in wild-type and caspase-1 deficient mice (FIGS. 4J and K, respectively), suggesting that processing and activation of IL-1β can also be achieved independently or in the absence of caspase-1, or that the detection of IL-1beta does not discriminate between inactive pro-IL-1beta and active mature IL-1beta.

To ascertain the relative roles of IL-1α and IL-1β to neutrophilic inflammation, we administered an anti-IL-1α or anti-IL-1β blocking antibody, or an isotype control antibody to cigarette smoke-exposed mice. While anti-IL-1α intervention abrogated smoke-induced neutrophilia (FIG. 5A), neither anti-IL-1β blockade nor administration of an isotype control impacted cigarette smoke-induced inflammation. These data suggest a critical role for IL-1α in mediating cigarette smoke-induced inflammation.

Since IL-1α significantly attenuated neutrophil recruitment to the lung of smoke-exposed mice, we assessed if neutrophil recruiting chemokines were preferentially decreased by blocking of IL-1α. We observed significantly increased expression of CXCL-1 RNA and protein following cigarette smoke-exposure (FIGS. 5 B and C, respectively). Anti-IL-1α, but not anti-IL-10 decreased CXCL-1 RNA and protein expression in smoke-exposed mice. Isotype antibody delivery did not alter transcript or protein expression levels. Furthermore, CXCL-2, CXCL-10 and CXCL-5 gene expression, which increased following smoke-exposure, decreased following treatment with anti-IL-1α, but not IL-1β (FIG. 5F). Together, these data are consistent with the conclusion that the neutrophilic inflammation observed in smoke-exposed animals requires the expression of CXCL -1, -2, and -5, and the expression of these factors are attenuated by blockade of IL-1α, but not IL-1β.

As both IL-1α and IL-1β signal through the IL-1R1, we next examined whether IL-1αinhibition decreased expression of IL-1β. FIG. 5 shows significantly decreased IL-1β transcript and protein levels in cigarette smoke-exposed mice that received anti-IL-1α antibody (panels D and E, respectively). Similarly, we observed decreased expression of GM-CSF, a cytokine that has recently been implicated in cigarette smoke-induced inflammation. We also found that anti-IL-1α, but not IL-1β inhibition significantly decreased expression levels of the macrophage elastase MMP-12. These data demonstrate that IL-1α, but not IL-1β is critical for mediating the signals leading to the accumulation of neutrophils within the lung of smoke-exposed mice.

Animals.

BALB/c mice (6-8 wk old) were purchased from Charles River Laboratories (Montreal, Canada). C57BL/6, IL-1R1-deficient, and caspase-1-deficient mice were obtained from Jackson Laboratories (Bar Harbor, Me., USA). Mice were maintained under specific pathogen-free conditions in an access-restricted area, on a 12 hour light-dark cycle, with food and water provided ad libitum.

Cigarette Smoke Exposure.

Mice were exposed to cigarette smoke using the SIU-48 whole body smoke exposure system (Promech Lab AB, Vintrie, Sweden) as previously described. Briefly, mice were exposed to 12 2R4F reference cigarettes with filters removed (Tobacco and Health Research Institute, University of Kentucky, Lexington, Ky., USA) for a period of approximately 50 minutes. This protocol of smoke exposure has been validated and shown to achieve blood carboxyhaemoglobin and cotinine levels that are comparable to those found in regular human smokers. Control animals were exposed to room air only.

Administration of Antibodies.

Mice were injected intraperitoneally with 400 μg of anti-IL-1α(clone ALF161; R&D Systems, Burlington, Canada), anti-IL-1β (clone B122; R&D Systems), or Armenian hamster isotype control antibody (Jackson Immunoresearch, Burlington, Canada) 12 hours prior to the first smoke exposure, and then daily 1 hour following the second smoke exposure. Bioactivity of IL-1alpha and IL-1beta antibodies were confirmed in vitro (in addition to suppliers quality control steps) by demonstrating inhibition of IL-1 induced IL-6 release from bEnd-3 (mouse endothelial cell line) cells.

Collection and Measurement of Specimens.

Bronchoalveolar lavage (BAL) fluid was collected after filling lungs with 0.25 ml of ice-cold 1×PBS followed by 0.2 ml of 1×PBS. Total cell numbers were obtained using a haemocytometer. Cytospins were prepared for differential cell counts and stained with Hema 3 (Biochemical Sciences Inc., Swedesboro, N.J., USA). 300 cells were counted per cytospin and standard hemocytological criteria were used to classify mononuclear cells, neutrophils, and eosinophils.

Histological Analysis and Immunohistochemistry.

Following BAL of mouse lung, the left lobe was fixed at 30 cm H₂0 pressure with 10% formalin. Lungs were embedded in paraffin blocks and 4 μm thick cross-sections were generated. For the IL-1α and IL-1β stain, prior to the primary antibody incubation, Rodent M Block (Biocare Medical, Concord, Calif., USA) was added to each slide for 30 minutes, and then washed away with a Tris-buffered saline with 0.05% Tween-20 (TBS-T). 10 μg/ml of goat anti-mouse IL-1α and IL-1β (R&D Systems, Minneapolis, Minn., USA) were prepared in Ultra Antibody Diluent (Thermo Scientific, Rockford, Ill., USA) and incubated with the slides for 1 hour. A secondary goat polymer horse-radish peroxidase was used according to the manufacturer's instructions (BioCare Medical; Concord, Calif., USA).

RNA Extraction for Fluidigm Analysis.

RNA was extracted from a single mouse lobe using the Qiagen RNeasy Fibrous Tissue kit according to the manufacturer's protocol (Qiagen, Hilden, Germany). RNA was quantified and normalized, and RNA integrity was assessed by Agilent Bioanalyzer using the Agilent RNA 6000 Nano Kit (Agilent, Santa Clara, Calif., USA). cDNA generation was carried out with the Super Script III kit from Life Technologies utilizing the manufacturer's protocol (Life Technologies, Carlsbard, Calif., USA). Relative transcript expression was assessed using the Fluidigm Biomark Dynamic array loaded with probes for transcripts of interest as previously described.

ELISA and Meso Scale Discovery Analysis.

Enzyme-linked immunoassay kits for IL-1α and IL-1β were purchased from R&D Systems (Minneapolis, Minn., USA) and the assay carried out according to the recommended protocol. Multi-array platform cytokine detection of keratin-derived cytokine (KC) and IL-1β was done using the multi-array murine pro-inflammatory and Th1/Th2 cytokine panel detection systems developed by Meso Scale Discovery (MSD; Gaithersburg, Md., USA).

Data and Statistical Analysis.

Data were analyzed using Graphpad Prism Software version 5 (La Jolla, Calif., USA) and expressed as mean±SEM. Statistical analysis was performed with SPSS statistical software, version 17.0 (Chicago, Ill., USA). We assessed significance (p<0.05) using the SPSS Univariate General Linear Model, t-tests were subsequently performed for two-group comparisons or one-way ANOVA with a Dunnett post-hoc test for multiple group comparisons.

### Example 4

### IL-1 Receptor Expression on Radio-Resistant Stromal Cells is Essential for Cigarette Smoke-Induced Inflammation

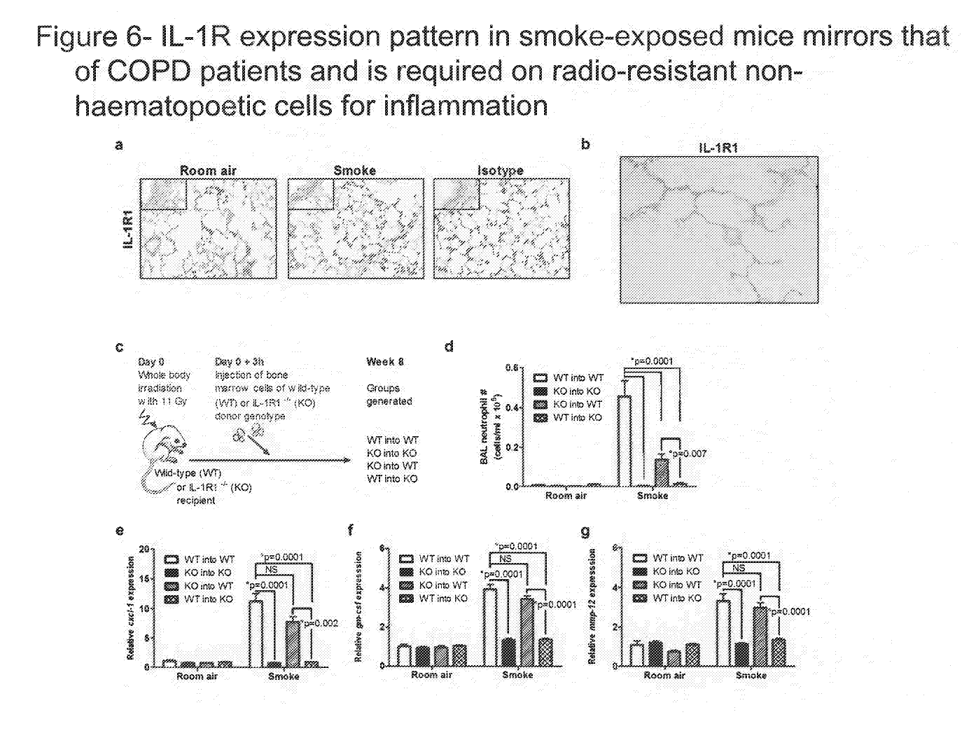

As can be seen by comparison of FIGS. 6A and B, tissue expression of IL-1R1 in smoke-exposed mice involves a similar population of resident cells to that seen in COPD patients.

To test the importance of crosstalk between hematopoietic and non-hematopoietic cells in the cigarette smoke-induced inflammation model of COPD, we generated IL-1R1-deficient bone marrow chimeric mice. Bone marrow cells from wild-type or IL-1R1-deficient mice were transferred intravenously to irradiated wild-type or IL-1R1-deficient recipient mice (FIG. 6C). Following 8 weeks of reconstitution, mice were exposed to cigarette smoke and various inflammatory parameters were assessed. Wild-type animals that received wild-type bone marrow cells (WT into WT) developed robust neutrophilia in response to cigarette smoke exposure (FIG. 6D); while no neutrophilia was observed in IL-1R1-deficient animals reconstituted with IL-1R1-deficient bone marrow cells (KO into KO). Chimeric mice, that resulted from the transfer of wild-type hematopoietic cells into irradiated IL-1R1-deficent mice (WT into KO), failed to demonstrate a neutrophilic response to smoke, suggesting that IL-1R1 expression on non-hematopoietic radio-resistant cells was essential for cigarette smoke-induced inflammation. Finally, transfer of IL-1R1-deficient hematopoietic cells into irradiated wild-type recipient mice (KO into WT) showed a significant, but partial reduction in cigarette smoke-induced neutrophilia.

We also investigated the expression of various genes, including, CXCL-1, GM-CSF, and MMP-12 (FIG. 6 E-G, respectively), all of which were decreased in IL-1R1 deficient animals reconstituted with IL-1R1 deficient bone marrow cells (KO into KO). Interestingly, while cigarette smoke-exposed WT into KO chimeric animals had significantly decreased gene expression, KO into WT animals did not—when compared to WT into WT control animals. These results support that IL-1R1 mediated activation of non-hematopoietic cells is a prerequisite for cigarette smoke-induced inflammation, while IL-1R1 expression on hematopoietic cells is required for maximal neutrophil infiltration. This is important since IL-1 alpha and beta upregulated in the lung would in theory act rapidly and locally on lung resident cells expressing IL-1R1 to induce inflammation. Without being bound by theory, these results may suggest that an IL-1R1 blocking strategy may be more effective than blocking soluble IL-1, and that blockade of IL-1R1 both in the lung and systemically would have additional benefit.

### Methods:

For immunochemistry for IL-1R1 staining in human sections, see example 12. Mouse immunochemistry essentially as for example 3, but with 5 μg/ml of goat anti-mouse IL-1R1 antibody (R&D Systems, Minneapolis, Minn., USA) incubated on the slides for 1 hour in place of anti-IL-1 alpha or beta antibodies.

Generation of IL-1R1-deficient Bone Marrow Chimeric Mice.

5 million C57BL/6 wild type or IL-1R1-deficient bone marrow cells were injected intravenously into irradiated (2 doses of 550Rads (11Gray total)) recipient C57BL/6 wild type (WT) or IL-1R1-deficient (knockout (KO)) mice. Recipient mice were on trimethoprim and sulfamethoxazole antibiotic-treated water one week prior to irradiation and two weeks following irradiation. Mice were allowed 8 weeks for reconstitution of hematopoietic bone marrow cells. Smoke administration was essentially as for Example 3.

The next examples relate to models of relevance to acute exacerbations of COPD (AECOPD)

### Example 5

### IL-1R1 Antagonist Inhibited LPS Mediated Inflammatory Cell Influx into Lung

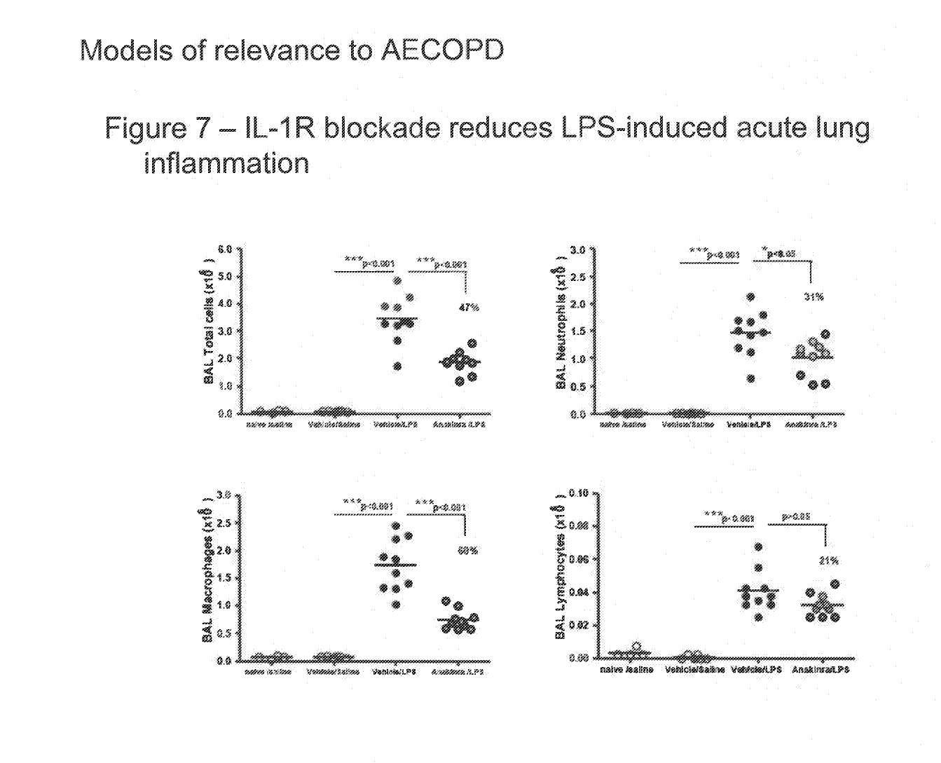

Lipopolysaccharide (LPS) is a component of bacterial cell walls of gram negative bacteria. These bacteria have been shown to be one trigger of acute exacerbations of COPD, and inhaled LPS-induced inflammation is one way to model such events. The effect of an IL-1R1 antagonist, anakinra, was examined in a mouse model of LPS mediated inflammatory cell influx into the lung. Anakinra inhibited LPS mediated inflammatory cell influx as measured by BAL total cells into the lung by 47% compared to control LPS treated mice (P<0.001) (FIG. 7).

### Methods:

Anakinra was delivered using an ALZET osmotic pump as described for acute smoke model, and was also administered to the mice 48 hours before the LPS administration. Mice were adult female Balb/c mice.

The mice were placed in a semi-open exposure inhalation box (max 10 mice) and were exposed once to aerosolised LPS—total inhalation session time 12 minutes. P. aeriginosa LPS was used at a concentration of 5 mg/ml and was aerosolised using a nebuliser (such as a PariStar Jet Star nebuliser), filled with 5 ml volume and flow from the nebuliser was 5 l/min (Pressure=2 bar). The control groups received the same procedure but with PBS. The mice were terminated 48 hours after LPS challenge using an i.p. injection of pentobarbital, the trachea was exposed and the lungs lavaged using room temperature PBS (without Ca or Mg) at 23 cm fluid pressure taking 2 minutes in and 1 minute out and then repeating the procedure. The BAL was then centrifuged—the cell pellet was analysed using standard automated cell counting and differential cell counting. The lungs were also removed for homogenisation for mRNA analysis or cytokine/mediator analysis. The significance of differences between groups was calculated using Student's T test with one-tailed distribution and two-sample unequal variance. Limits for p-values using unequal variance T-test: p<0.05.

### Example 6

### IL-1R1 Modulates Responses of Lung Epithelial Cell Lines and Primary Normal Human Brochial Epithelial Cells to Rhinoviral Infection

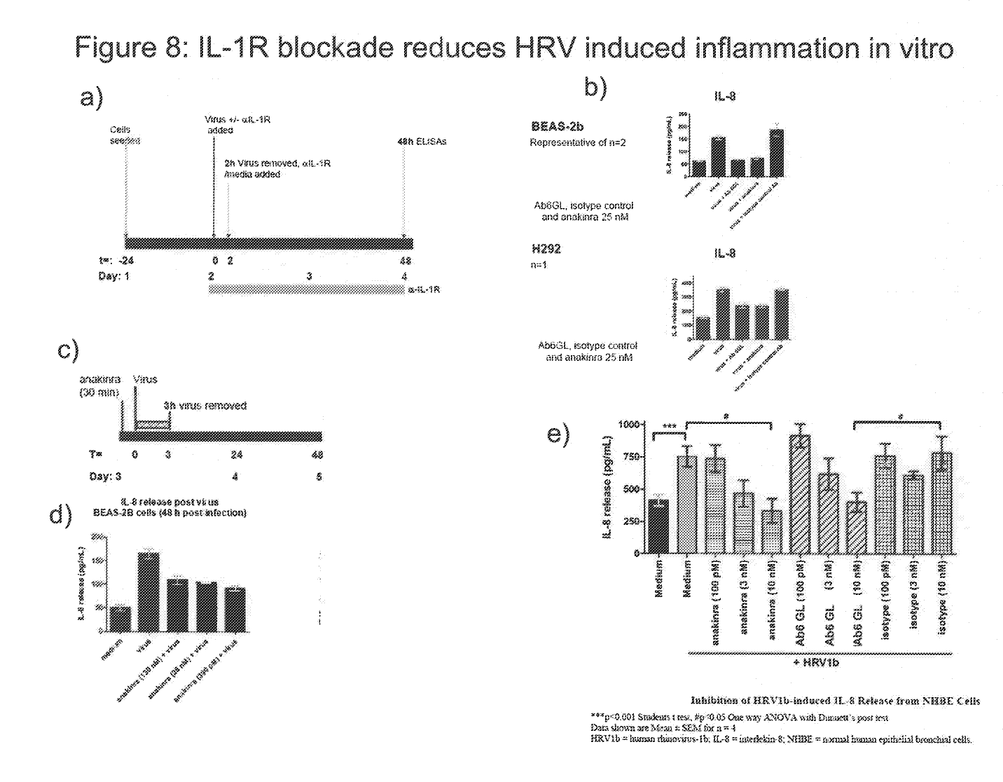

Human rhinovirus is a common virus which has been implicated in acute exacerbation of COPD (AECOPD). COPD patients have been shown to have an exacerbated response to rhinovirus. To investigate the role of IL-1 in human rhinovirus (HRV)-mediated inflammatory response, PEG purified HRV14 was used to infect BEAS-2b/H292 cells (human cells available from the ATCC) while those cells were being exposed to an IL-1R antagonist (FIG. 8A). For Methods see example 8. A prototypic inflammatory mediator IL-8 (CXCL-1) was examined after treatment and HRV14 infection of the cells (FIG. 8). IL-8 levels were reduced with both antibody 6, germ-lined and anakinra (FIG. 8B), but not by isotype control antibody. The concentration of anakinra used on the cells was 25 nM. An alternative protocol was additionally used as shown in FIG. 8C, and the results are provided in 8D. Anakinra was tested at 3 concentrations, all of which reduced IL-8 release in response to HRV14 in BEAS-2B cells. BEAS-2B and H292 cells are epithelial cell lines, so additionally this response was analysed in more physiologically relevant primary normal human bronchal epithelial cells, sourced from Lonza (FIG. 8E). Human rhinovirus infection (HRV1b) of normal human bronchial epithelial (NHBE) cells resulted in increased IL-8 release into culture medium, measured 48 hours after infection. Antibody 6, germlined (Ab6GL; 10 nM) significantly inhibited the response to rhinovirus when compared to rhinovirus+isotype control. Ab6GL inhibited the response to a similar extent as anakinra (Kineret®), which was used as a positive control. Anakinra (10 nM) had a significant effect on IL-8 production from epithelial cells in response to rhinovirus infection, when compared to the rhinovirus alone group (FIG. 8E). Human rhinovirus-1b (minor group virus) was used in these experiments so that comparisons could be made between effects of IL-1R1 blockade in vitro and in vivo (see Example 7): Human rhinovirus-1b is able to infect mice whereas major group HRVs (such as HRV14) are not able to infect mice. In vitro effects of IL-1 blockade on minor and major group rhinovirus (HRV14) induced IL-8 production showed similar trends. This illustrates that IL-1R1 blockade reduces the pro-inflammatory response to human rhinovirus in vitro. This attribute is useful in normalising COPD exacerbated response to rhinovirus infection.

### Example 7

### IL-1R1 Blockade Reduces Virus Induced Inflammation to HRV in Acute Mouse Model

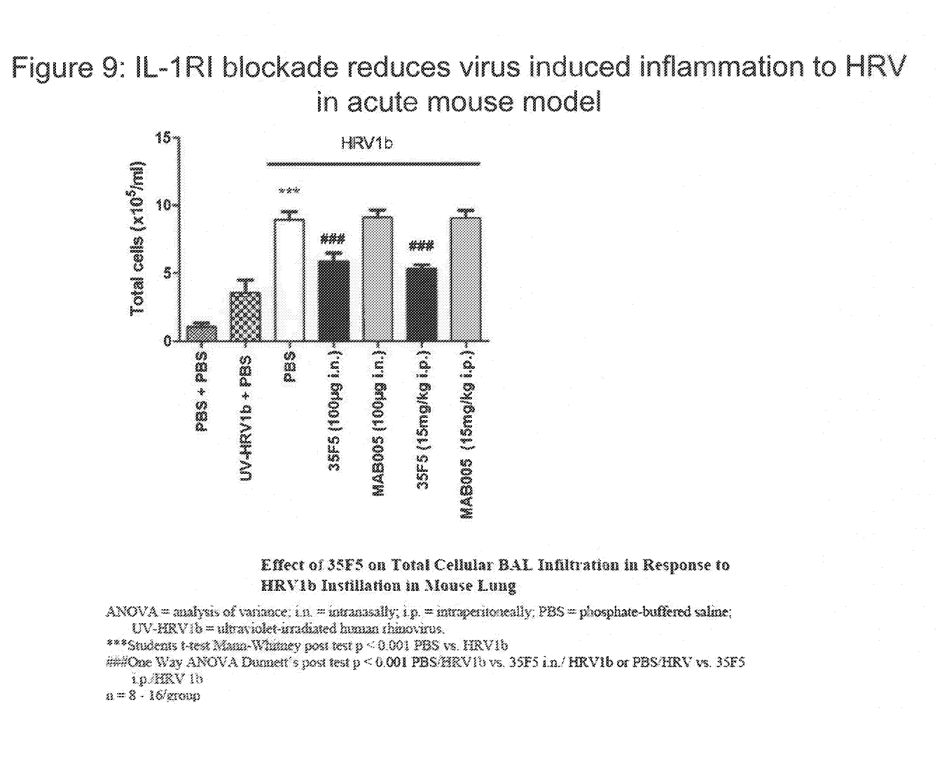

To investigate whether anti-IL-1R1 could abrogate the proinflammatory neutrophilic response to virus, the commercially available anti-mouse IL-1R1 antibody 35F5 (described above) was employed in a murine HRV challenge model. The minor group serotype HRV1b has been shown to infect mouse epithelial cells and induce an acute inflammation in mouse lungs and was used in this study. In order to test whether anti-IL-1R1 inhibition has similar anti-inflammatory effects in a viral challenge model in vivo, the ability of systemically and intranasally administered 35F5 to reduce HRV-induced cellular inflammation in lungs was determined. Human rhinovirus-1b intranasal administration (purified virus, 107 plaque forming units [pfu]/mL) significantly increased total cell and neutrophil counts in BAL 24 hours after viral administration. Viral load was not measured due to the acute nature of the model. Ultraviolet-irradiated rhinovirus produced a reduced inflammatory response as measured by cellular infiltration into BAL, showing that a significant portion of the response is dependent on intact virus. The anti-mouse IL-1R1 antibody 35F5 or an isotype control (Rat IgG1; MAB005) was given as a single dose of 15 mg/kg intraperitoneally or 100 μg intranasally to mice 24 hours prior to intranasal challenge with purified HRV1b. Cellular infiltrate into the BAL of animals was measured 24 hours after virus instillation. 35F5 significantly reduced total cellular infiltration (FIG. 9) and influx of neutrophils into the BAL of mice in response to HRV1b challenge. Reduction of neutrophilic inflammation in response to virus is likely beneficial in COPD where there is underlying chronic inflammation which is exacerbated by viral infection.

### Example 8

### IL-1R1 Blockade Reduces Inflammation in Response to Smoke and Smoke+Virus in Epithelial Cells

The inflammatory response of epithelial cell in vitro was measured in response to smoke conditioned medium, or smoke conditioned medium and virus. The smoke conditioned medium was generated by bubbling cigarette smoke through tissue culture (TC) medium, and is referred to later in this example as ‘smoke’ or ‘smoke treatment’ of the cells. One cigarette with the filter removed bubbled through 25 mL medium is equal to 100% smoked medium. Cigarette smoke treated medium was titrated for IL-8 release and cell confluence on BEAS-2b cells. 20% smoked medium was used for all experiments as it induced pro-inflammatory cytokine release without significant cell death.

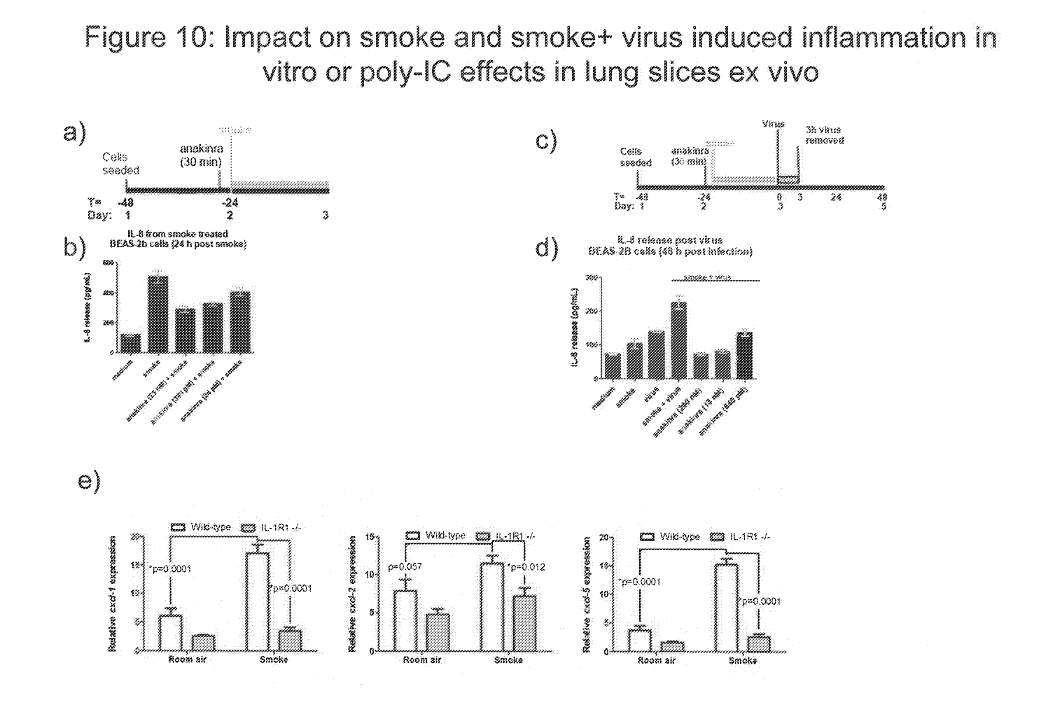

To examine the role of IL-1R in smoke and virus induced inflammation, cells were first smoke treated with a pre-treatment of IL-1R antagonist, and then as required, infected with HRV virus with another pre-treatment of IL-1R antagonist, anakinra. (FIGS. 10A and 10C). The experiment was performed four times with different concentrations of anakinra (as shown in the figures).

Anakinra treatment resulted in partial inhibition of smoke-induced IL-8 response (FIG. 10B). Smoke and virus stimuli were additive in terms of IL-8 response Anakinra treatment post smoke and virus exposure inhibited the combined smoke and virus IL-8 response (FIG. 10D). Concentration dependent and complete inhibition was achieved. These results indicate that treatment with an IL-1R antagonist can inhibit the inflammatory response to viral infection, as well as that of a combination of smoke and viral infection, as assessed by inhibition of IL-8 response.

### Methods (Relating to Both Example 6 and Example 8):

Cells used for epithelial smoke and virus work were BEAS-2B cells obtained from ATCC (catalogue number CRL-9609) and grown as per suppliers instructions, or H292s from ECACC (catalogue no 91091815 NCI-H292) also grown according to suppliers instructions.

A lit cigarette (no filter) was connected by tubing to a falcon tube (50 ml capacity) containing tissue culture medium which was supported in a glass flask. A peristaltic pump drew the smoke through the tubing and into the tissue culture medium. The waste smoke was drawn into a beaker of detergent. The whole procedure was performed within a fume cupboard to protect the operator and other users of the lab. The procedure was therefore not sterile. In order to maintain sterility as much as possible, the falcon tube containing medium was placed into the conical flask using forceps that have been wiped with 70% ethanol. The pipette inserted through the bung that delivers the smoke to the tissue culture medium was replaced each time and was wiped down with 70% ethanol immediately before the procedure. The falcon tube was recovered with forceps and the lid replaced as soon as possible. The smoked medium was then diluted and placed on to cells as soon as possible, preferably within an hour of completion of the smoking procedure [n.b. the medium did not contain serum for the smoke extract procedure]. Additionally, antibiotics (gentomycin) were included in the standard culture/assay medium for these cells. The base medium for this cell line (BEBM) along with all the additives were obtained from Lonza/Clonetics Corporation as a kit: BEGM, Kit Catalog No. CC-3170.

Cells were exposed to rhinovirus (major group HRV14 prepared and titred using Hela-Ohio cells by standard practice and either used crude or with PEG-precipitation of virus), as per schedules shown.

Cells were seeded onto collagen coated flat clear bottomed plates and were incubated at 37° C. 5% CO₂ and left to adhere overnight. Medium was removed from wells and replaced with media+/−anakinra in 150 ul (anakinra at 2× final concentration). Cells were incubated for 30 minutes at 37° C. 5% CO₂. Smoke medium was prepared as described (smoke extract can be prepared using Kentucky research grade cigarettes). Smoke extract was diluted to 40% with media and then added to cells in 150 ul without removal of media+/−anakinra. Some cells had media alone as controls. These were incubated 24 hours. 200 ul of supernatants were removed and frozen for later cytokine analysis. Remaining media was removed and discarded. Anakinra or media was replaced onto cells in 100 ul and the virus was added 30 minutes later at a dilution determined by titres of virus stock on HeLa OHIO cells to determine equivalent activity for each batch made in an additional 100 ul . Cells were incubated for 3 hours at 37° C. 5% CO₂. All media was then removed from cells, anakinra or fresh media was added to the cells and incubated for a further 48 hours at 37° C. 5% CO₂.

IL-8 was measured in supernatants using ELISA kit (R&D Systems Duoset DY208) according to manufacturer's instructions and using R&D recombinant protein as standards for the assays.

### Example 9

### IL-1R1 Deficiency in Smoke-Exposed Precision Cut Lung Slices (PCLS) Attenuates Lung Resident Responses to Viral Stimulus

In this example, we assessed whether similar mechanisms may underlie the differential response of the smoke-exposed lung to viral challenge. We generated precision cut lung slices (PCLS) from the lungs of room air- and smoke-exposed wild-type and IL-1R1-deficient mice. PCLS were stimulated ex vivo with the dsRNA ligand, polyinosinic polycytidylic acid (polyl:C), and expression of key mediators were assessed. We observed a significantly greater induction in response to polyl:C stimulation of neutrophil recruiting chemokines, CXCL-1 and CXCL-5, and a modest increase in CXCL-2 from PCLS generated from smoke-exposed wild-type compared to room air-exposed controls (FIG. 10E). All transcripts measured were significantly attenuated in viral mimic-stimulated smoke-exposed IL-1R1-deficient PCLS. Collectively, these data demonstrate a role for lung resident cells in promoting smoke-induced inflammation and support a role for the IL-1R1 in the differential response of the smoke-exposed lung to viral infection.

Methods:

Precision Cut Lung Slicing and Culture.

Lungs were sliced using a modification to a standard protocol that has previously been described in Bergner et al., 2002, Journal of General Physiology 119: 187-198. Such modifications are further described in Khan et al., 2007, European Respir Journal 30: 691-700. Briefly, lungs were inflated with approximately 1.4 ml of agarose (type VH-A low gelling temperature; Sigma Aldrich, St. Louis, Mo., USA) that was warmed to 37° C. and prepared to a concentration of 2% in Hank's buffered saline solution (HBSS), supplemented with N-2-hydroxyethlypiperazine-N′-2-ethanesulphonic acid (HEPES) (0.2M, pH 7.4). Subsequently, 0.2 ml of air was injected into the lung in order to flush the agarose-HBSS solution out of the conducting airways. The agarose was allowed to gel by cooling the lung to 4° C. for 15 minutes. The lung lobes were dissected away and a flat surface was cut on the lobe parallel and caudal to the main bronchus. The lung lobes were maintained in an ice-cold 1×HBSS solution prior to and during slicing. 120 μm thick slices were generated using a vibratome (Leica; model VT 1000S, Richmond Hill, Canada) at 4° C. Approximately 40 slices were isolated from each mouse lung.

Lung slices were subsequently transferred to and cultured in Dulbecco's Modified Eagles Medium (DMEM)/F12 (Gibco, Burlington, Canada) supplemented with 35 μg/mL-Ascorbic Acid (Sigma-Aldrich, Oakville, Canada), 5 μg/ml Transferin (Gibco, Burlington, Canada), 2.85 μg/ml Insulin (Sigma-Aldrich, Oakville, Canada), and 3.25 ng/ml Selenium (atomic absorption standard solution; Sigma-Aldrich, Oakville, Canada). The solution was filter-sterilized using a 0.22 μm pore filter. The DMEM/F12 solution was further supplemented with 250 ng/ml Amphotericin B (Sigma-Aldrich, Oakville, Canada) and 1% penicillin/streptomycin. The medium was changed every 1 hour for the first 3 hours of culture in order to remove any remaining agarose and cell debris from the lung slice culture. Lung slices were stimulated the next day for 6 hours with 100 ug/ml of dsRNA mimetic polyinosinic-polycytidylic acid (GE Healthcare, Mississauga, Canada) that was reconstituted in phosphate buffered saline or were left untreated. Samples were collected in RNA later (Ambion, Austin, Tex., USA) and preserved at −80° C. until extraction of RNA.

RNA Extraction and Real-Time Quantitative RT PCR for Precision Cut Lung Slices.

Lung slices were collected and placed into 200 μl of RNAlater (Qiagen, Mississauga, ON, Canada), and stored at −80° C. until needed. RNA was extracted from the lung slices according to the animal tissues protocol from the RNEasy Kit (Qiagen, Mississauga, ON, Canada). Optional on-column DNase digestion was performed. RNA was quantified using the Agilent 2100 Bio-analyzer (Agilent Technologies, Mississauga, ON, Canada). The quantity and integrity of isolated RNA was determined using the Agilent 2100 Bioanalyzer (Agilent, Palo Alto, Calif., USA). Subsequently, 100 ng of total RNA was reverse-transcribed using 100 U of Superscript II (Invitrogen, Burlington, Canada) in a total volume of 20 μL. Random hexamer primers were used to synthesize cDNA at 42° C. for 50 minutes, followed by 15 minutes incubations at 70° C. Real-time quantitative RT-PCR was performed in triplicate, in a total volume of 25 μl, using a Universal PCR Master Mix (Applied Biosystems, Foster City, Calif., USA). Primers for CXCL-1, CXCL-2, CXCL-5, GAPDH, along with FAM-labeled probes were purchased from Applied Biosystems. PCR was performed using the ABI PRISM 7900HT Sequence Detection System using the Sequence Detector Software version 2.2 (Applied Biosystems, Foster City, Calif., USA). Data were analyzed using the delta, delta Ct method. Briefly, gene expression was normalized to the housekeeping gene (GAPDH) and expressed as fold change over the control group (room air control, mock).

### Example 10

### IL-1R1 Deficiency and IL-1Alpha Antibody Blockade Attenuates Exaggerated Inflammation in a Model of H1N1 Influenza Virus Infection of Smoke-Exposed Mice

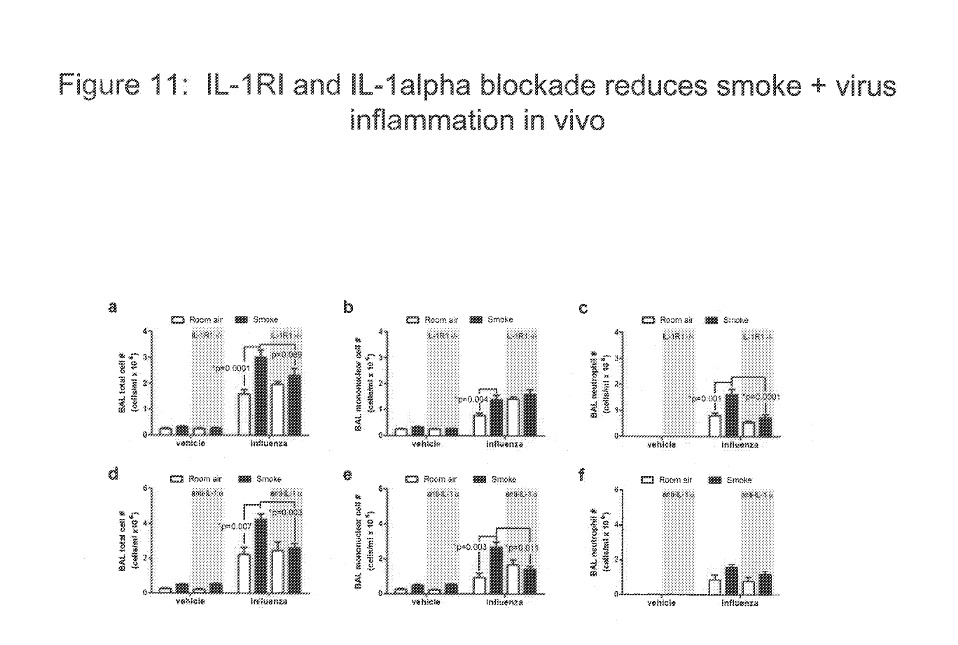

Having established the importance of IL-1α in mediating signals via the IL-1R1 for the induction of smoke-induced inflammation, and given the role that resident cells of the smoke-exposed lung were shown to play in the response to viral insult (see Example 9) we sought next to assess if these mechanisms underlie the exacerbated inflammatory response observed following viral infection in vivo. Wild-type and IL-1R1-deficient mice were exposed to cigarette smoke and subsequently infected with a H1N1 influenza virus. An exacerbated inflammatory response was observed in the BAL of cigarette smoke-exposed wild-type mice following viral infection compared to virally-infected room air control mice (FIG. 11A). While an IL-1R1 deficiency modestly attenuated (p=0.089) total BAL inflammation in smoke-exposed influenza-infected mice, neutrophilia was significantly decreased in these animals compared to wild-type controls (FIG. 11C). These data suggest that an IL-1R1 dependent mechanism contributes to exacerbation of the inflammatory response in smoke-exposed mice following viral infection.

While an IL-1R1 deficiency could lessen exaggerated inflammatory responses in smoke-exposed influenza-infected animals, we hypothesized that IL-1α would play a predominate role in promoting this response. To test this, we injected animals daily with the anti-IL-1α or isotype antibodies during the course of cigarette smoke-exposure and viral infection. An exacerbated response to influenza A virus, in cigarette smoke-exposed mice, was observed 5 days post-infection (FIG. 11D). Anti-IL-1α neutralization markedly attenuated BAL total inflammation, with the effect significantly impacting mononuclear cells, but not neutrophils (FIGS. 11E and F, respectively). Taken together these data support the conclusion that therapies aimed at blocking IL-1α/IL-1R1 may be beneficial during periods of disease instability, particularly during COPD exacerbation.

Methods: Essentially as for smoke models described in example 3. Influenza infected animals also received daily intraperitoneal injections during the course of infection.

Influenza Infection.

Anesthetized mice were intranasally infected with 50 PFU of a mouse-adapted H1N1 influenza A (A/FM/1/47-MA) virus in 35 μl of 1× phosphate-buffered saline (PBS) vehicle. Control animals received 35 μl of PBS vehicle. A/FM/1/47-MA is a fully sequenced, plaque-purified preparation that is biologically characterized with respect to mouse lung infections. Animals were not exposed to cigarette smoke on the day of viral delivery or for the entire course of the viral infection.

For the viral studies, prior to BAL one lobe from the right lung was removed for determination of viral titre. The remainder of the right lung was preserved in RNA later (Ambion, Austin, Tex., USA), and the left lung lobe was inflated with formalin for histological assessment.

The next examples are of particular relevance to human COPD.

### Example 11

### COPD Patient Exacerbation Correlates with Increased IL-1 Alpha and IL-1Beta Levels

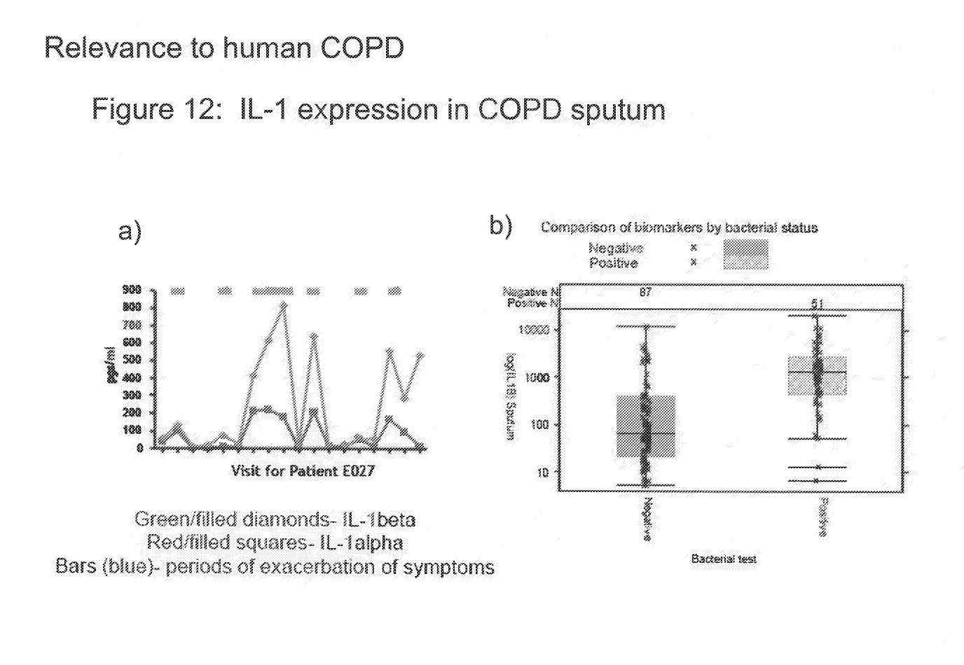

Sputum measurements of COPD human patients were analyzed for IL-1 alpha and IL-1beta levels in comparison with exacerbation timing over an extended period of time. Sputum was processed using PBS processing and not with DTT processing in order to least perturb the sputum cytokine content. In this patient, both IL-1alpha and IL-1beta were upregulated on exacerbation of COPD (FIG. 12A). The periods of exacerbation strongly correlated with increased IL-1alpha and IL-1beta levels.

In a different patient subset, correlation of bacterial status and IL1-beta was also analyzed. IL-1beta was significantly higher in patients with a positive test for bacteria in their sputum (FIG. 12B).

### Example 12

### IL-1Alpha and IL-1Beta are Increased in the Lung of COPD Patients

In this example we examined expression of IL-1α and IL-1β in the lung of GOLD I & II COPD patients. Lung section biopsies stained positively for both IL-1α and 13 (FIGS. 13A and B, respectively). There was a significantly greater number of IL-1α and 13 positive cells observed in biopsy samples taken from GOLD I & II COPD patients compared to non-COPD controls (FIG. 13C).

Given the importance of lung structural cells in initiating inflammatory responses (see example 4), we assessed IL-1α and β staining of lung epithelium in COPD patients compared to non-COPD controls. While IL-1α was not increased in the epithelium of COPD patients compared to non-COPD controls, IL-1β staining was significantly increased (p<0.0001) (FIGS. 13D and E, respectively). Levels of IL-1α and β recovered from the sputum of COPD patients were significantly correlated (p<0.0001) during stable disease, at the onset of exacerbation (prior to additional treatment), and 7 and 35 days post-exacerbation (FIGS. 13F-I). Correlation between IL-1α and β was strongest at 7 days post exacerbation. In a subset of patients, levels of IL-1α and β were increased at exacerbation compared to levels measured during the stable disease visit. Taken together, these data support the conclusion that IL-1 signaling plays a role, not only in stable COPD, but also during episodes of acute exacerbation and that blockade of IL-1R1 represents a successful strategy to treat exacerbations.

Methods: Human Lung Biopsies and Sputum Samples.

Lung sections were obtained from biopsy samples taken from GOLD I (n=3, 1 male and 2 females; current smoker, n=3; mean±SD of FEV1/FVC %=60±8) and GOLD II (n=6, 4 males and 2 females; current smoker, n=2; mean±SD of FEV1/FVC %=56±10) COPD patients. Biopsy data from these two groups were combined. Data were compared with non-COPD materials obtained from cancer lobectomy from anatomically normal lobe regions. Sputum samples were obtained from COPD patients at enrollment during stable disease, at onset of exacerbation, and 7 days and 35 days post-on set of exacerbation. Exacerbation was defined as increase in two major (dyspnoea, sputum volume, or sputum purulence) symptoms or one major and one minor (cough, wheeze, sore throat, nasal discharge, fever) symptom over a 48 hour period. Patients were given a normal standard of care under the presenting circumstances, and sputum samples were taken at the discretion of the study investigator.

For human expression of IL-1α, IL-1β, and IL-1R1 antigen retrieval was performed by incubating sections in 0.2% trypsin/0.2% CaCl₂ in distilled H₂O at 37° C. for 10 minutes. Endogenous peroxidase activity was blocked using 6% H₂0₂ for 10 minutes. To block non-specific binding of the secondary antibody, slides were incubated with 20% normal rabbit or goat serum for 20 minutes. Excess serum was removed and slides were incubated with either IL-1αrabbit anti-human antibody (Abeam, 9614, 2.5 μg/ml), IL-1β rabbit anti-human antibody (Abeam, 2105, 10 μg/ml) or IL-1R1 goat anti-human antibody (R&D Systems, Ab-269-NA, 10 μg/ml) or either rabbit or goat IgG negative control for 1 hour. Slides were incubated with biotinylated rabbit anti-goat secondary (1:200) or swine anti-rabbit secondary (1:200) antibody for 20 minutes. Two antigen detection protocols were employed on the human tissue sets: 1) Strep ABComplex/HRP (Dako) for 20 minutes at room temperature, 2×10 minutes buffer wash and DAB applied for 1 minute. 2) Strep ABComplex/AP (Dako) for 30 minutes at room temperature, 2×10 minutes buffer wash and Fuchsin Substrate-Chromagen System (Dako) for 5 minutes. Slides were counterstained with haematoxylin (Sigma). Positive cells were counted from two separate biopsy samples from each patient taken approximately 10 μm apart. A 250 mm² graticule was aligned to the basement membrane and cells counted in the lamina propria in 3 adjacent regions.

### Sequences:

The following provides sequence information for certain antibodies.

(SEQ ID NO: 1)
Antibody 6 VH amino acid sequence = Glu Val Gln Leu Leu Glu Ser Gly Gly Gly Leu Val Gln
Pro Gly Gly Ser Leu Arg Leu Ser Cys Ala Ala Ser Gly Phe Thr Phe Ser Ser Tyr Ala Met Ser Trp
Val Arg Gln Ala Pro Gly Lys Gly Leu Glu Trp Val Ser Ala Ile Ser Gly Ser Gly Gly Ser Thr Tyr
Tyr Ala Asp Ser Val Lys Gly Arg Phe Thr Ile Ser Arg Asp Asn Ser Lys Asn Thr Leu Tyr Leu
Gln Met Asn Ser Leu Arg Ala Glu Asp Thr Ala Val Tyr Tyr Cys Ala Lys Pro Leu Tyr Tyr Tyr
Asp Glu Gln Tyr Gly Val Val Tyr Asp Ala Phe Val Trp Gly Arg Gly Thr Met Val Thr Val Ser
Ser
(SEQ ID NO: 2)
Antibody 6 heavy chain CDR1 = Ser Tyr Ala Met Ser
(SEQ ID NO: 3)
Antibody 6 heavy chain CDR2 = Ala Ile Ser Gly Ser Gly Gly Ser Thr Tyr Tyr Ala Asp Ser Val
Lys Gly
(SEQ ID NO: 4)
Antibody 6 heavy chain CDR3 = Pro Leu Tyr Tyr Tyr Asp Glu Gln Tyr Gly Val Val Tyr Asp
Ala Phe Val
(SEQ ID NO: 5)
Antibody 6 VL amino acid sequence = Gln Ser Val Leu Thr Gln Pro Pro Ser Val Ser Gly Ala
Pro Gly Gln Arg Val Thr Ile Ser Cys Thr Gly Ser Ser Ser Asn Ile Gly Ala Gly Tyr Asp Val His
Trp Tyr Gln Gln Leu Pro Gly Thr Ala Pro Lys Leu Leu Ile Tyr Gly Asp Thr His Arg Pro Ser Gly
Val Pro Asp Arg Phe Ser Gly Ser Lys Ser Gly Thr Ser Ala Ser Leu Val Ile Ala Gly Leu Gln Ala
Glu Asp Glu Ala Asp Tyr Tyr Cys Gln Ser Tyr Asp Thr Val Arg Leu His His Val Phe Gly Gly
Gly Thr Lys Leu Thr Val Leu
(SEQ ID NO: 6)
Antibody 6 light chain CDR1 = Thr Gly Ser Ser Ser Asn Ile Gly Ala Gly Tyr Asp Val His
(SEQ ID NO: 7)
Antibody 6 light chain CDR2 = Gly Asp Thr His Arg Pro Ser
(SEQ ID NO: 8)
Antibody 6 light chain CDR3 = Gln Ser Tyr Asp Thr Val Arg Leu His His Val
(SEQ ID NO: 9)
Antibody 6 VH-germlined = Glu Val Gln Leu Leu Glu Ser Gly Gly Gly Leu Val Gln Pro Gly
Gly Ser Leu Arg Leu Ser Cys Ala Ala Ser Gly Phe Thr Phe Ser Ser Tyr Ala Met Ser Trp Val Arg
Gln Ala Pro Gly Lys Gly Leu Glu Trp Val Ser Ala Ile Ser Gly Ser Gly Gly Ser Thr Tyr Tyr Ala
Asp Ser Val Lys Gly Arg Phe Thr Ile Ser Arg Asp Asn Ser Lys Asn Thr Leu Tyr Leu Gln Met
Asn Ser Leu Arg Ala Glu Asp Thr Ala Val Tyr Tyr Cys Ala Lys Pro Leu Tyr Tyr Tyr Asp Glu
Gln Tyr Gly Val Val Tyr Asp Ala Phe Val Trp Gly Arg Gly Thr Leu Val Thr Val Ser Ser
(SEQ ID NO: 10)
Antibody 6 VL-germlined = Gln Ser Val Leu Thr Gln Pro Pro Ser Val Ser Gly Ala Pro Gly
Gln Arg Val Thr Ile Ser Cys Thr Gly Ser Ser Ser Asn Ile Gly Ala Gly Tyr Asp Val His Trp Tyr
Gln Gln Leu Pro Gly Thr Ala Pro Lys Leu Leu Ile Tyr Gly Asp Thr His Arg Pro Ser Gly Val Pro
Asp Arg Phe Ser Gly Ser Lys Ser Gly Thr Ser Ala Ser Leu Ala Ile Thr Gly Leu Gln Ala Glu Asp
Glu Ala Asp Tyr Tyr Cys Gln Ser Tyr Asp Thr Val Arg Leu His His Val Phe Gly Gly Gly Thr
Lys Leu Thr Val Leu
(SEQ ID NO: 31)
Gln Val Gln Leu Val Glu Ser Gly Gly Gly Val Val Gln Pro Gly Arg Ser Leu Arg Leu Ser Cys
Ala Ala Ser Gly Phe Thr Phe Ser Asn Tyr Gly Met His Trp Val Arg Gln Ala Pro Gly Lys Gly
Leu Glu Trp Val Ala Gly Ile Trp Asn Asp Gly Ile Asn Lys Tyr His Ala His Ser Val Arg Gly
Arg Phe Thr Ile Ser Arg Asp Asn Ser Lys Asn Thr Leu Tyr Leu Gln Met Asn Ser Pro Arg Ala
Glu Asp Thr Ala Val Tyr Tyr Cys Ala Arg Ala Arg Ser Phe Asp Trp Leu Leu Phe Glu Phe
Trp Gly Gln Gly Thr Leu Val Thr Val Ser Ser
CDR1, CDR2, and CDR3 are underlined and bolded.
(SEQ ID NO: 32)
CDR1 = NYGMH
(SEQ ID NO: 33)
CDR2 = GIWNDGINKYHAHSVRG
(SEQ ID NO: 34)
CDR3 = ARSFDWLLFEF
Antibody 26F5-VL (light chain variable domain)
(SEQ ID NO: 35)
Glu Ile Val Leu Thr Gln Ser Pro Ala Thr Leu Ser Leu Ser Pro Gly Glu Arg Ala Thr Leu Ser Cys
Arg Ala Ser Gln Ser Val Ser Ser Tyr Leu Ala Trp Tyr Gln Gln Lys Pro Gly Gln Ala Pro Arg
Leu Leu Ile Tyr Asp Ala Ser Asn Arg Ala Thr Gly Ile Pro Ala Arg Phe Ser Gly Ser Gly Ser
Gly Thr Asp Phe Thr Leu Thr Ile Ser Ser Leu Glu Pro Glu Asp Phe Ala Val Tyr Tyr Cys Gln
Gln Arg Ser Asn Trp Pro Pro Leu Thr Phe Gly Gly Gly Thr Lys Val Glu Ile Lys
CDR1, CDR2, and CDR3 are underlined and bolded.
(SEQ ID NO: 36)
CDR1 = RASQSVSSYLA
(SEQ ID NO: 37)
CDR2 = DASNRAT
(SEQ ID NO: 38)
CDR3 = QQRSNWPPLT
Antibody 27F2-VH (heavy chain variable domain)
(SEQ ID NO: 39)
Gln Val Gln Leu Val Glu Ser Gly Gly Gly Val Val Gln Pro Gly Arg Ser Leu Arg Leu Ser Cys
Ala Val Ser Gly Phe Thr Phe Ser Asn Tyr Gly Met His Trp Val Arg Gln Ala Pro Gly Lys Gly
Leu Glu Trp Val Ala Ala Ile Trp Asn Asp Gly Glu Asn Lys His His Ala Gly Ser Val Arg Gly
Arg Phe Thr Ile Ser Arg Asp Asn Ser Lys Asn Thr Leu Tyr Leu Gln Met Asn Ser Leu Arg Ala
Glu Asp Thr Ala Val Tyr Tyr Cys Ala Arg Gly Arg Tyr Phe Asp Trp Leu Leu Phe Glu Tyr
Trp Gly Gln Gly Thr Leu Val Thr Val Ser Ser
CDR1, CDR2, and CDR3 are underlined and bolded.
(SEQ ID NO: 40)
CDR1 = TFSNYGMH
(SEQ ID NO: 41)
CDR2 = AIWNDGENKHHAGSVRG
(SEQ ID NO: 42)
CDR3 = GRYFDWLLFEY
Antibody 27F2-VL (light chain variable domain)
(SEQ ID NO: 35)
Glu Ile Val Leu Thr Gln Ser Pro Ala Thr Leu Ser Leu Ser Pro Gly Glu Arg Ala Thr Leu Ser Cys
Arg Ala Ser Gln Ser Val Ser Ser Tyr Leu Ala Trp Tyr Gln Gln Lys Pro Gly Gln Ala Pro Arg
Leu Leu Ile Tyr Asp Ala Ser Asn Arg Ala Thr Gly Ile Pro Ala Arg Phe Ser Gly Ser Gly Ser
Gly Thr Asp Phe Thr Leu Thr Ile Ser Ser Leu Glu Pro Glu Asp Phe Ala Val Tyr Tyr Cys Gln
Gln Arg Ser Asn Trp Pro Pro Leu Thr Phe Gly Gly Gly Thr Lys Val Glu Ile Lys
CDR1, CDR2, and CDR3 are underlined and bolded.
(SEQ ID NO: 36)
CDR1 = RASQSVSSYLA
(SEQ ID NO: 37)
CDR2 = DASNRAT
(SEQ ID NO: 38)
CDR3 = QQRSNWPPLT
Antibody 27F2-VH (heavy chain variable domain)
(SEQ ID NO: 39)
Gln Val Gln Leu Val Glu Ser Gly gly Gly Val Val Gln Pro Gly Arg Ser Leu Arg Leu Ser Cys
Ala Val Ser Gly Phe Thr Phe Ser Asn Tyr Gly Met His Trp Val Arg Gln Ala Pro Gly Lys Gly
Leu Glu Trp Val Ala Ala Ile Trp Asn Asp Gly Glu Asn Lys His His Ala Gly Ser Val Arg Gly
Arg Phe Thr Ile Ser Arg Asp Asn Ser Lys Asn Thr Leu Tyr Leu Gln Met Asn Ser Leu Arg Ala
Glu Asp Thr Ala Val Tyr Tyr Cys Ala Arg Gly Arg Tyr Phe Asp Trp Leu Leu Phe Glu Tyr
Trp Gly Gln Gly Thr Leu Val Thr Val Ser Ser
CDR1, CDR2, and CDR3 are underlined and bolded.
(SEQ ID NO: 40)
CDR1 = TFSNYGMH
(SEQ ID NO: 41)
CDR2 = AIWNDGENKHHAGSVRG
(SEQ ID NO: 42)
CDR3 = GRYDWLLFEY
Antibody 27F2-VL (light chain variable domain)
(SEQ ID NO: 35)
Glu Ile Val Leu Thr Gln Ser Pro Ala Thr Leu Ser Leu Ser Pro Gly Glu Arg Ala Thr Leu Ser Cys
Arg Ala Ser Gln Ser Val Ser Ser Tyr Leu Ala Trp Tyr Gln Gln Lys Pro Gly Gln Ala Pro Arg
Leu Leu Ile Tyr Asp Ala Ser Asn Arg Ala Thr Gly Ile Pro Ala Arg Phe Ser Gly Ser Gly Ser
Gly Thr Asp Phe Thr Leu Thr Thr Ile Ser Ser Leu Glu Pro Glu Asp Phe Ala Val Tyr Tyr Cys Gln
Gln Arg Ser Asn Trp Pro Pro Leu Thr Phe Gly Gly Gly Thr Lys Val Glu Ile Lys
CDR1, CDR2, and CDR3 are underlined and bolded.
(SEQ ID NO: 36)
CDR1 = RASQSVSSYLA
(SEQ ID NO: 37)
CDR2 = DASNRAT
(SEQ ID NO: 38)
CDR3 = QQRSNWPPLT
Antibody 15C4-VH (heavy chain variable domain)
(SEQ ID NO: 43)
Glu Val Gln Leu Met Gln Ser Gly Ala Glu Val Lys Lys Pro Gly Glu Ser Leu Lys Ile Ser Cys
Lys Gly Ser Gly Tyr Ser Phe Ser Phe His Trp Ile Ala Trp Val Arg Gln Met Pro Gly Lys Gly
Leu Glu Trp Met Gly Ile Ile His Pro Gly Ala Ser Asp Thr Arg Tyr Ser Pro Ser Phe Gln Gly
Gln Val Thr Ile Ser Ala Asp Asn Ser Asn Ser Ala Thr Tyr Leu Gln Trp Ser Ser Leu Lys Ala Ser
Asp Thr Ala Met Tyr Phe Cys Ala Arg Gln Arg Glu Leu Asp Tyr Phe Asp Tyr Trp Gly Gln
Gly Thr Leu Val Thr Val Ser Ser
CDR1, CDR2, and CDR3 are underlined and bolded.
(SEQ ID NO: 44)
CDR1 = FHWIA
(SEQ ID NO: 45)
CDR2 = IIHPGASDTRYSPSFQG
(SEQ ID NO: 46)
CDR3 = QRELDYFDY
Antibody 15C4-VL (light chain variable domain)
(SEQ ID NO: 47)
Glu Ile Val Leu Thr Gln Ser Pro Asp Phe Gln Ser Val Thr Pro Lys Glu Lys Val Thr Ile Thr Cys
Arg Ala Ser Gln Ser Ile Gly Ser Ser Leu His Trp Tyr Gln Gln Lys Pro Asp Gln Ser Pro Lys
Leu Leu Ile Lys Tyr Ala Ser Gln Ser Phe Ser Gly Val Pro Ser Arg Phe Ser Gly Ser Gly Ser
Gly Thr Asp Phe Thr Leu Thr Ile Asn Ser Leu Glu Ala Glu Asp Ala Ala Ala Tyr Tyr Cys His
Gln Ser Ser Ser Leu Pro Leu Thr Phe Gly Gly Gly Thr Lys Val Glu Ile Lys
CDR1, CDR2, and CDR3 are underlined and bolded.
(SEQ ID NO: 48)
CDR1 = RASQSIGSSLH
(SEQ ID NO: 49)
CDR2 = YASQSFS
(SEQ ID NO: 50)
CDR3 = HQSSSLPLT

### INCORPORATION BY REFERENCE

All publications and patents mentioned herein are hereby incorporated by reference in their entirety as if each individual publication or patent was specifically and individually indicated to be incorporated by reference.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A method of reducing airway inflammation in a patient in need thereof, wherein said patient is a patient having chronic obstructive pulmonary disease (COPD) exacerbation, comprising administering to said patient an effective amount of a composition comprising an antibody that specifically binds to and inhibits IL-IR1 thereby reducing airway inflammation in said patient.

2. A method of reducing IL-1α signaling in a patient in need thereof, wherein said patient is a patient having chronic obstructive pulmonary disease (COPD) exacerbation, comprising administering to said patient an effective amount of a composition comprising an antibody that specifically binds to and inhibits IL-1R1 thereby reducing IL-1α signaling in said patient.

3. The method of claim 1 or 2, wherein the antibody is a recombinant antibody that inhibits binding of IL-1R1 to IL-1α, IL-1β, or both IL-1α, and IL-1β.

4-5. (canceled)

6. The method of claim 1, wherein reducing airway inflammation includes a reduction in neutrophil influx into a lung.

7-8. (canceled)

9. The method of claim 1 or 2, wherein said recombinant antibody is a human antibody

10. The method of claim 1 or 2, or wherein the method is part of a therapeutic regimen for treating COPD.

11. The method of claim 10, wherein the therapeutic regimen for treating COPD comprises administration of steroids.

12-13. (canceled)

14. The method of claim 1 or 2, wherein the COPD exacerbation is caused by smoke.

15. The method of claim 1 or 2, wherein the antibody specifically binds to IL-1R1 with a K_{D} of 50 pM or less when measure by Biacore™.

16-22. (canceled)

23. A method of treating COPD exacerbation in a patient in need thereof, wherein said patient is a patient having COPD exacerbation due to viral or bacterial infection, comprising administering to said patient an effective amount of a composition comprising an antibody that specifically binds to and inhibits binding of IL-IR1 to IL-1α.

24-25. (canceled)

26. The method of claim 23, wherein treating COPD exacerbation comprises reducing airway inflammation.

27. The method of claim 23, wherein treating COPD exacerbation comprises reducing neutrophil influx into a lung.

28-29. (canceled)

30. The method of claim 23, wherein said antibody inhibits binding of IL-1R1 to IL-1α, IL-1β, or both IL-1α and IL-Iβ.

31. The method of claim 23, wherein said antibody is a human antibody

32-35. (canceled)

36. The method of claim 23, further comprising the administration of steroids.

37-38. (canceled)

39. The method of claim 23, wherein said antibody specifically binds to IL-1R1 with a KD of 50 pM or less when measure by Biacore™.

40. The method of claim 23, wherein said recombinant antibody is antibody 6 or an antibody having the CDRs of antibody 6.

41. The method of claim 23, wherein said recombinant antibody competes with IL-IRa, for binding to IL-1R1.

42-45. (canceled)
